(19) United States
(12) Patent Application Publication
(10) Pub. No.: US 2024/0245822 A1
(43) Pub. Date: Jul. 25, 2024

(54) STERILIZATION MANAGEMENT SYSTEM, STERILIZATION MANAGEMENT METHOD AND INFORMATION PROCESSING APPARATUS

(71) Applicant: CANON KABUSHIKI KAISHA, Tokyo (JP)

(72) Inventor: TOSHIFUMI KITAMURA, Shizuoka (JP)

(73) Assignee: CANON KABUSHIKI KAISHA, Tokyo (JP)

(21) Appl. No.: 18/624,358

(22) Filed: Apr. 2, 2024

Related U.S. Application Data
(63) Continuation of application No. PCT/JP2022/031042, filed on Aug. 17, 2022.

(30) Foreign Application Priority Data
Oct. 20, 2021 (JP) 2021-171903

(51) Int. Cl. A61L 2/28; G06K 7/14; G06K 19/07

(52) U.S. Cl. CPC A61L 2/28; G06K 7/1417; G06K 19/0723; A61L 2202/122; A61L 2202/14

(57) ABSTRACT

A first wireless device (WD) is installed at a known position, and a second WD is attached to a target of a sterilization process. A reading apparatus attempts reading identification information from WDs, and measures a relative amount of movement from a reference position using a self-localization technique. An estimation unit of a system estimates a position of the target based on the relative amount of movement of the reading apparatus from a time point when the first identification information has been read from the first WD to a time point when the second identification information has been read from the second WD. A database stores position information representing an estimated position of the target and sterilization-related information. The estimated position of the target selected based on the sterilization-related information is notified to a user based on the position information.

### CROSS-REFERENCE TO RELATED APPLICATIONS

This application is a Continuation of International Patent Application No. PCT/JP2022/031042, Aug. 17, 2022, which claims the benefit of Japanese Patent Application No. 2021-171903, filed Oct. 20, 2021, both of which are hereby incorporated by reference herein in their entirety.

### BACKGROUND OF THE INVENTION

### Field of the Invention

The present disclosure relates to a sterilization management system, a sterilization management method and an information processing apparatus.

### Background Art

Typically, in medical institutions, sterilization processes for sterilizing instruments used in cures or treatments of patients have been performed. The sterilization processes include putting targets inside a sterilizer and exposing the targets to high pressure steam or a sterilization agent (for example, hydrogen peroxide gas or ethylene gas) or irradiating targets with ultraviolet light for a sufficient amount of time. Also, recently, the general public has become more conscious of hygiene, and from the need to combat infectious diseases, sterilization processes are being used on various items in business and social activities. The sterilization processes in such cases may be performed by a simpler method of spraying or applying a sanitizer liquid, leaving the target to soak in sanitizer liquid in a container, or the like. In any case, if there are many targets for sterilization processes, it may become difficult to know which targets have been sterilized and managing timings of the sterilization processes for individual targets may become complicated.

PTL 1 and PTL 2 disclose systems for assisting in the management of sterilization process deadlines. The system of PTL 1 manages sterilization-related information including, for example, sterilization process dates for sterilization targets that need to be re-sterilized due to expiration of effective periods of sterilization or other reasons in a database, generates statistical information, and provides the statistical information to users. In the system of PTL 2, a two-dimensional barcode or non-contact tag representing individual information is attached to a set of sterilization targets, and readers that can read the individual information are placed at various locations such as at a sterilizer and at a storing location. The reader at the sterilizer reads the individual information when the sterilization process is performed and associates the information on the sterilization process with the set of sterilization targets. The reader at the storing location reads the individual information of the set to be stored and associates the information of the storing location with the set of sterilization targets.

### CITATION LIST

### Patent Literature

PTL 1: Japanese Patent Laid-Open No. 2021-13697
PTL 2: Japanese Patent Laid-Open No. 2021-108029

With a technique, such as that of the system of PTL 2, in which readers are fixed at some restricted locations, there may be no way to know whether a sterilization process should be performed for an item that exists at a location without a reader, and it may also be difficult to find an item that requires a sterilization process.

The present invention has been made in consideration of the aforementioned situation and provides a mechanism that can realize more efficient sterilization management of sterilization targets that may move to any place within a space for users' activities.

### SUMMARY OF THE INVENTION

According to an aspect, there is provided a sterilization management system, including: a first wireless device installed at a known position and storing first identification information; a second wireless device attached to a target of a sterilization process and storing second identification information; a reading apparatus that moves together with a mobile object, the reading apparatus being capable of reading, from a wireless device, identification information stored in the wireless device, and capable of measuring a relative amount of movement from a reference position using a self-localization technique; a position estimation unit configured to estimate a position of the target based on the relative amount of movement of the reading apparatus from a first point in time at which the first identification information has been read from the first wireless device by the reading apparatus to a second point in time at which the second identification information has been read from the second wireless device by the reading apparatus; a database that stores position information representing an estimated position of the target and sterilization-related information for the target; and a notification unit configured to, when the target is selected based on the sterilization-related information, notify a user of a position at which the target is estimated to exist based on the position information stored in the database. There is also provided a corresponding sterilization management method and an information processing apparatus.

Further features of the present invention will become apparent from the following description of exemplary embodiments with reference to the attached drawings.

### BRIEF DESCRIPTION OF THE DRAWINGS

FIG. 1 is a schematic view illustrating an example of a configuration of a sterilization management system according to an embodiment.

FIG. 2 is a block diagram illustrating an example of a configuration of a tag reader according to an embodiment.

FIG. 3 is a block diagram illustrating an example of a configuration of a management server according to an embodiment.

FIG. 4 is an explanatory diagram illustrating an example of a configuration of a target table according to an embodiment.

FIG. 5A is an explanatory diagram illustrating an example of a configuration of an area table according to an embodiment.

FIG. 5B is an explanatory diagram illustrating an example of a configuration of a sterilizer table according to an embodiment.

FIG. 5C is an explanatory diagram illustrating an example of a configuration of a reader table according to an embodiment.

FIG. 6A is an explanatory diagram illustrating an example of a configuration of a movement amount table according to an embodiment.

FIG. 6B is an explanatory diagram illustrating an example of a configuration of a tag detection table according to an embodiment.

FIG. 7 is an explanatory diagram illustrating an example of a configuration of a sterilization process table according to an embodiment.

FIG. 8A is a first part of an explanatory diagram for explaining an example of a scenario related to sterilization of a sterilization target.

FIG. 8B is a second part of the explanatory diagram for explaining an example of a scenario related to sterilization of a sterilization target.

FIG. 8C is a third part of the explanatory diagram for explaining an example of a scenario related to sterilization of a sterilization target.

FIG. 9 is an explanatory diagram for explaining association of a sterilization target with a sterilization process in the scenario of FIGS. 8A to 8C.

FIG. 10A is a first part of an explanatory diagram for explaining movement and detection of a sterilization target, following the scenario of FIGS. 8A to 8C.

FIG. 10B is a second part of the explanatory diagram for explaining movement and detection of a sterilization target, following the scenario of FIGS. 8A to 8C.

FIG. 11 is a first explanatory diagram for explaining an example of a graphical user interface (GUI) that assists sterilization management.

FIG. 12 is a second explanatory diagram for explaining an example of a GUI that assists sterilization management.

FIG. 13 is a third explanatory diagram for explaining an example of a GUI that assists sterilization management.

FIG. 14 is a fourth explanatory diagram for explaining an example of a GUI that assists sterilization management.

FIG. 15 is a fifth explanatory diagram for explaining an example of a GUI that assists sterilization management.

FIG. 16 is a block diagram illustrating an example of a configuration of a sterilizer according to an embodiment.

FIG. 17 is a flowchart illustrating an example of a flow of a data transmission process performed by a tag reader.

FIG. 18 is a flowchart illustrating an example of a flow of a data update process performed by a management server.

FIG. 19 is a flowchart illustrating a first example of a flow of a notification process performed by a management server.

FIG. 20 is a flowchart illustrating a second example of a flow of a notification process performed by a management server.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate.

Furthermore, in the attached drawings, the same reference numerals are given to the same or similar configurations, and redundant description thereof is omitted.

### 1. System Overview

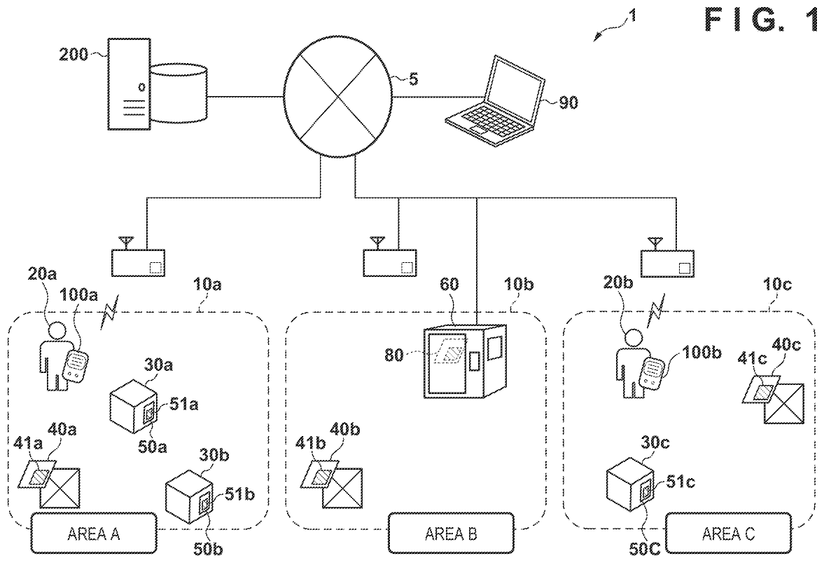

FIG. 1 is a schematic view illustrating an example of a configuration of a sterilization management system 1 according to an embodiment. The sterilization management system 1 is a system that assists management of sterilization of a plurality of items. In the following descriptions, items subject to sterilization processes are referred to as sterilization targets (or targets, simply). The management of sterilization may be performed in order to cause users to carry out sterilization in a timely manner or to prevent users from using a target that has not been appropriately sterilized based on information such as statuses regarding whether sterilization is effective for each sterilization target, and deadlines of sterilization processes, for example. In the present embodiment, for such purposes, the sterilization management system 1 maintains position information of each sterilization target in addition to sterilization-related information.

FIG. 1 illustrates three areas 10a, 10b and 10c that are preset in the sterilization management system 1. Each of these areas 10a, 10b and 10c may be a building, a floor in a building, a section set in a floor, a room, or a certain outdoor region.

There exists a user 20a in the area 10a. The user 20a moves freely across areas while carrying a tag reader 100a. There exists a user 20b in the area 10c. The user 20b moves freely across areas while carrying a tag reader 100b. In this specification, the expression that a user carries a certain target should broadly encompass various modes in which the user moves together with the target (for example, moves in a state where he or she holds or wears the target, etc.).

There exist items 30a and 30b in the area 10a. There exists an item 30c in the area 10c. Each of the items 30a, 30b and 30c is a sterilization target. Each of the items 30a, 30b and 30c may move from an area to another area through regular activities of the users 20a, 20b (and other users).

Note that, in the following descriptions, the areas 10a, 10b and 10c are collectively referred to as areas 10 by omitting the trailing letters from the reference signs when they do not need to be distinguished from each other. The same applies to the users 20 (users 20a, 20b), the items 30 (items 30a, 30b, 30c), and the tag readers 100 (tag readers 100a, 100b), as well as other elements. The number of areas 10, the number of users 20 and the number of items 30 under management of the system are not limited to the example illustrated in FIG. 1 but may be any numbers.

A sterilizer 60 with a door that can be opened and closed is installed in the area 10b. For example, when the item 30a needs to be sterilized, the user 20a carries the item 30a to the area 10b, puts the item 30a into the sterilizer 60, and activates the sterilizer 60 to start a sterilization process. In situations where it is required to ensure that sterilization is completed such as sterilization of medical instruments, an indicator such as a chemical indicator or biological indicator is placed in the sterilizer 60 together with the sterilization target, and the completion of the sterilization process is confirmed based on a change (for example, a change in color) of the indicator. In other situations, such an indicator may not be used. The sterilizer 60 comprises an operation unit that accepts a user operation, a communication interface that communicates with the management server 200, and a display unit that displays information, in addition to circuitry that controls a sterilization process such that a sterilization target is sterilized under appropriate process conditions. Note that, though an example where a sterilization target is sterilized inside a sterilizer 60 is mainly described in the present embodiment, sterilization may be performed using a spray type or disinfection tank type instrument in another embodiment. FIG. 1 shows only one sterilizer 60 for simplicity of explanation, however, several different sterilizers 60 may be available, of course.

The sterilization management system 1 makes use of wireless devices, which are also referred to as tags, in order to assist sterilization management. A position tag is a first type of wireless device (first wireless devices) which is installed in each of the areas 10 under management of the sterilization management system 1. FIG. 1 shows a position tag 40a installed in the area 10a, a position tag 40b installed in the area 10b, and a position tag 40c installed in the area 10c. The installation position of each position tag 40 is known to the sterilization management system 1. An item tag is a second type of wireless device (second wireless devices) which is attached to each of items 30 that are sterilization targets. FIG. 1 shows item tags 50a, 50b, and 50c that are attached to the items 30a, 30b, and 30c, respectively.

In the present embodiment, each of the tags such as the position tags 40 and item tags 50 is assumed to be a passive-type radio frequency identification (RFID) tag (a passive tag). A passive tag is composed of: a small integrated circuit (IC) chip with an embedded memory; and an antenna, and has identification information for identifying the tag and some other information stored in the memory. In this specification, identification information is simply referred to as an ID, and identification information for identifying a tag is referred to as a tag ID. It should be noted that the tag ID may be considered as information for identifying an object to which the tag is attached. In the example of FIG. 1, the position tags 40a, 40b and 40c have specific tag IDs (first identification information) 41a, 41b and 41c embedded in the tags, respectively. The item tags 50a, 50b and 50c have specific tag IDs (second identification information) 51a, 51b and 51c embedded in the tags, respectively. The IC chip of a passive tag operates by utilizing energy of an electromagnetic wave emitted from a tag reader, and modulates the information such as the tag ID and some other information stored in the memory into an information signal to transmit (send back) the information signal from the antenna.

In the present embodiment, a sterilization tag 80 is further installed on the sterilizer 60. The sterilization tag 80 is a third type of wireless device (third wireless device) storing a tag ID (third identification information) that identifies the sterilizer 60. The sterilization tag 80 may be a passive tag, too. For example, the housing and door of the sterilizer 60 may be made of metal, in which case the tag ID of the sterilization tag 80 will not be read by an external tag reader 100 unless the door is opened.

It should be noted that, in another embodiment, each tag may be an active-type RFID tag. If each tag actively (for example, periodically) transmits information to its vicinity by utilizing power from a built-in battery, such a tag may be called a beacon tag. In a further embodiment, each tag may be a wireless device which sends back information in response to a signal from a reader in accordance with Near Field Communication (NFC) protocol or Bluetooth (registered trademark) protocol, for example. Each tag may have any name such as an IC tag, an IC card, or a responder.

The sterilization management system 1 includes a terminal apparatus 90, tag readers 100, and a management server 200. The terminal apparatus 90, the tag readers 100, and the management server 200 are connected to a network 5. The network 5 may be a wired network, a wireless network, or any combination thereof. Examples of the network 5 may include the Internet, an intranet, and a cloud network.

The terminal apparatus 90 is a user terminal used by a user 20 or a manager of the sterilization management system 1. The terminal apparatus 90 may be a general-purpose terminal such as a personal computer (PC) or a smartphone, or a dedicated terminal specialized for a sterilization management purpose. The terminal apparatus 90 may be portable or stationary. The terminal apparatus 90 typically comprises an input device that receives user inputs, a communication interface that communicates with other apparatuses (for example, the management server 200), and a display device that displays information. As an example, the terminal apparatus 90 is used by a user 20 or a manager when browsing information provided from the management server 200.

The tag reader 100 is a reading apparatus that is capable of reading, from wireless devices such as RFID tags, information stored in the wireless devices. For example, the tag reader 100 can detect a position tag 40 by reading a tag ID from the position tag 40. Likewise, the tag reader 100 can detect an item 30 by reading the tag ID 51 from the item tag 50 attached to the item 30. In addition, in the present embodiments, the tag reader 100 is capable of measuring a relative amount of movement from a reference position using a self-localization technique. The attempt of tag reading and the measurement of movement amount by the tag reader 100 may be continuously performed without any explicit instruction from the user 20 while the tag reader 100 is operating. The tag reader 100 provides the management server 200 with the results of such attempt of tag reading and measurement of movement amount. The tag reader 100 may be capable of communicating with the management server 200 directly or indirectly via a certain relay apparatus (for example, a terminal carried by the user 20, or the like). An example of a particular configuration of the tag reader 100 will be further described below.

Note that, though an example where a user 20 carries a tag reader 100 is mainly described, the tag reader 100 is not limited to this example and may move among areas together with any type of a mobile object. Mobile objects may include humans, vehicles, carts, wheelchairs, robots and drones, for example. A mobile object may move automatically or autonomously, or may move by humans.

The management server 200 is an information processing apparatus that manages the above-described position information, sterilization-related information, and other information in a database. The management server 200 may be implemented as an application server, a database server, or a cloud server by using a high-end general-purpose computer, for example. The management server 200 receives data indicating the results of tag reading and measurement of movement amount from tag readers 100, and updates the database based on the received data. In addition, the management server 200 provides the user 20 with information regarding sterilization targets (for example, via the terminal apparatus 90) in response to an inquiry from the user 20. An example of a particular configuration of the management server 200 will be further described below. Though a single management server 200 is illustrated in FIG. 1, the functions of the management server 200, which will be described in detail below, may be provided by a single apparatus or by physically-separate multiple apparatuses which operate in conjunction with each other. In addition, though an example where the management server 200 maintains a database will be described in the present embodiment, an apparatus other than the management server 200 may maintain a part or all of the database. For example, a part of data may be maintained by a wireless device or a tag reader 100.

Though FIG. 1 depicts the terminal apparatus 90 and the tag reader 100 as separate apparatuses, there may be provided an integrated apparatus that has both functionalities of the terminal apparatus 90 and the tag reader 100. The terminal apparatus 90 may be carried by a user 20 and may relay communication between the tag reader 100 and the management server 200. Moreover, the functions of the management server 200 described in the present specification may be realized within the terminal apparatus 90.

### 2. Configuration Example of Tag Reader

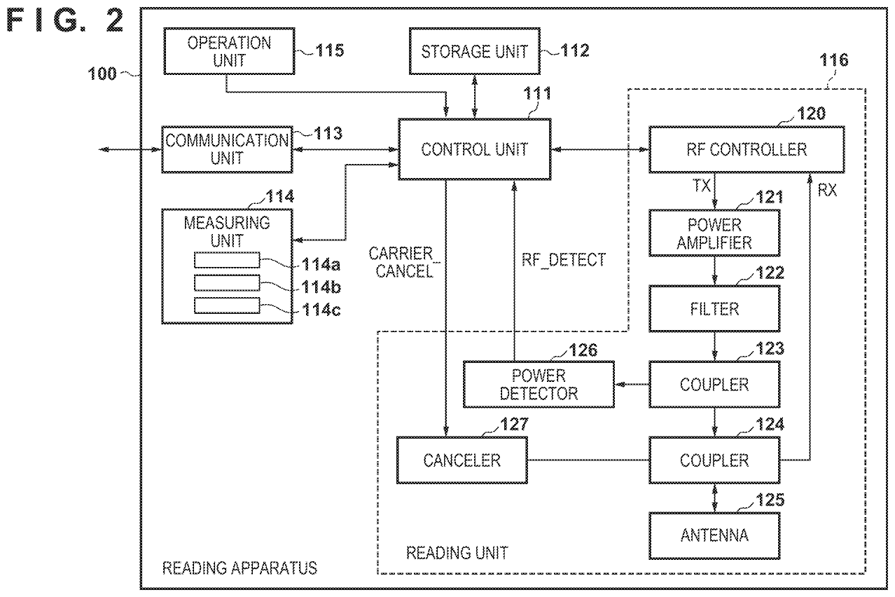

FIG. 2 is a block diagram illustrating an example of a configuration of the tag reader 100 according to an embodiment. With reference to FIG. 2, the tag reader 100 comprises a control unit 111, a storage unit 112, a communication unit 113, a measuring unit 114, an operation unit 115, and a reading unit 116.

The control unit 111 consists of a memory to store computer programs, and one or more processors (for example, central processing units (CPUs)) to execute the computer programs. The control unit 111 controls overall functionality of the tag reader 100 described in this specification. For example, the control unit 111 causes the reading unit 116 to perform reading from an RFID tag within a tag reading range, and causes the storage unit 112 to temporarily store the read information and the time of the reading as reading result data. In parallel to the reading from RFID tags, the control unit 111 also causes the measuring unit 114 to measure a relative amount of movement of the tag reader 100, and the storage unit 112 to store measurement result data indicating the measurement result. Then, the control unit 111 transmits, to the management server 200 via the communication unit 113, the reading result data and the measurement result data stored in the storage unit 112 together with the reader identification information (also referred to as a reader ID) of the tag reader 100.

The storage unit 112 may include any kind of storage medium such as a semiconductor memory (a read only memory (ROM), a random access memory (RAM), or the like), an optical disk, or a magnetic disk, for example. In the present embodiment, the storage unit 112 stores the above-described reading result data, measurement result data, and the reader ID of the tag reader 100.

The communication unit 113 is a communication interface for the tag reader 100 to communicate with the management server 200. For example, the communication unit 113 may be a wireless local area network (WLAN) interface that communicates with a WLAN access point, or a cellular communication interface that communicates with a cellular base station. Alternatively, the communication unit 113 may be a connection interface (e.g. a Bluetooth (registered trademark) interface or a universal serial bus (USB) interface) for connection with a relay apparatus.

The measuring unit 114 is a unit that is capable of measuring an amount of movement of the tag reader 100. In the present embodiment, the measuring unit 114 uses the self-localization technique, also referred to as pedestrian dead reckoning (PDR) to measure a relative amount of movement of the tag reader 100 from a certain reference position, and outputs the measured amount of movement to the control unit 111. The reference position of measurement of the relative amount of movement may be, for example, the position of the tag reader 100 at the time when the tag reader 100 is activated. For example, the measuring unit 114 includes three-axis acceleration sensor 114a, gyro sensor 114b, and geomagnetic sensor 114c. The three-axis acceleration sensor 114a measures acceleration applied to the tag reader 100 in the device coordinate system that is specific to the tag reader 100, and outputs first sensor data. The gyro sensor 114b measures an angular speed of the tag reader 100, that is a change in attitude of the tag reader 100, and outputs second sensor data. The geomagnetic sensor 114c measures an orientation of the tag reader 100 in the real space, and outputs third sensor data. The measuring unit 114 can measure the relative amount of movement of the tag reader 100 based on these pieces of sensor data by converting the direction of the acceleration of the tag reader 100 into a direction in a coordinate system of the real space to integrate the converted acceleration. The relative amount of movement of the tag reader 110 output from the measuring unit 114 to the control unit 111 may be a two-dimensional vector in a horizontal plane or a three-dimensional vector that includes a component of height direction as well.

As described below, in the present embodiment, the positional coordinates of the installation position of each position tag 40 are known and registered in a database. Therefore, the current (positional coordinates of) absolute position of the tag reader 100 can be estimated based on the relative amount of movement of the tag reader 100 from the time point where it detected a position tag 40 to the current time point, and the known positional coordinates of that position tag 40. In the present embodiment, an example where the management server 200 estimates a position of the tag reader 100 is mainly described, however, the control unit 111 or the measuring unit 114 of the tag reader 100 may access the database to estimate the position of the tag reader 100. Note that a measuring apparatus (which is carried by the user 20, for example) that is separate from the tag reader 100 may measure a relative amount of movement using PDR, instead of the tag reader 100 including the measuring unit 114. In that case, the tag reader 100 may receive measurement result data indicating a relative amount of movement via a communication link with the measuring apparatus.

The operation unit 115 receives an operation by the user 20. The operation unit 115 includes physical input devices such as a button, a switch, or a lever disposed on a housing of the tag reader 100, for example. The operation unit 115 receives an operation by the user 20 through an input device, and outputs an operation signal to the control unit 111. In addition, the operation unit 115 may include an audio input interface such as a microphone.

The reading unit 116 is a unit that is capable of reading, from various RFID tags under management of the sterilization management system 1, information stored in the tags. With reference to FIG. 2, the reading unit 116 includes an RF controller 120, a power amplifier 121, a filter 122, a first coupler 123, a second coupler 124, an antenna 125, a power detector 126, and a canceler 127. The RF controller 120 outputs a transmission signal (for example, a signal modulated in the UHF band) from a TX terminal to the power amplifier 121 in accordance with control by the control unit 111. The power amplifier 121 amplifies the transmission signal input from the RF controller 120 to output it to the filter 122. The amplification rate of the transmission signal here may be controllable in variable manner, and a higher amplification rate will enhance an output strength of an electromagnetic wave emitted from the tag reader 100. The filter 122 may be a low-pass filter, for example, and filters out unnecessary frequency components from the transmission signal amplified by the power amplifier 121. The first coupler 123 distributes the transmission signal that has passed the filter 122 to the coupler 124 and the power detector 126. The second coupler 124 outputs the transmission signal input from the first coupler 123 to the antenna 125, and outputs a received signal input from the antenna 125 to the RF controller 120. The antenna 125 transmits the transmission signal input from the coupler 124 to the air as an electromagnetic wave. Further, the antenna 125 receives a signal that has been sent back from an RFID tag that exists within the reading range of the tag reader 100 in response to the transmission signal, and outputs the received signal to the coupler 124. The power detector 126 detects a power level of the signal input from the first coupler 123, and outputs a signal ‘RF_DETECT’ indicative of the detected power level to the control unit 111. The canceler 127 receives a signal ‘CARRIER_CANCEL’ indicative of a power level of a carrier from the control unit 111. Then, the canceler 127 extracts an intended signal component of the received signal to be output to an RX terminal of the RF controller 120 by canceling the carrier component of the transmission signal based on the CARRIER_CANCEL. The RF controller 120 demodulates the signal input from the RX terminal to obtain a tag ID and other information sent back from the RFID tag, and outputs the obtained information to the control unit 111.

In the present embodiment, the reading unit 116 can attempt tag reading periodically (for example, once per second) without requiring any explicit command from a user. Data transmission from the communication unit 113 to the management server 200 can also be performed periodically (for example, every few seconds) or whenever the tag reading is done without requiring any explicit command from a user. The control unit 111 may exclude, from the data to be transmitted, the same record as the most recent record that has already been transmitted in a predetermined time period to omit redundant data transmission and reduce a communication load. Noted that, in another embodiment, one or both of an attempt of tag reading by the reading unit 116 and data transmission to the management server 200 may be performed in response to detecting a user input via the operation unit 115. In a case where the communication unit 113 performs communication with the management server 200 indirectly via a relay apparatus, the data transmission to the management server 200 may be performed only while there is an effective connection between the communication unit 113 and the relay apparatus.

### 3. Configuration Example of Management Server

### <3-1. Basic Configuration>

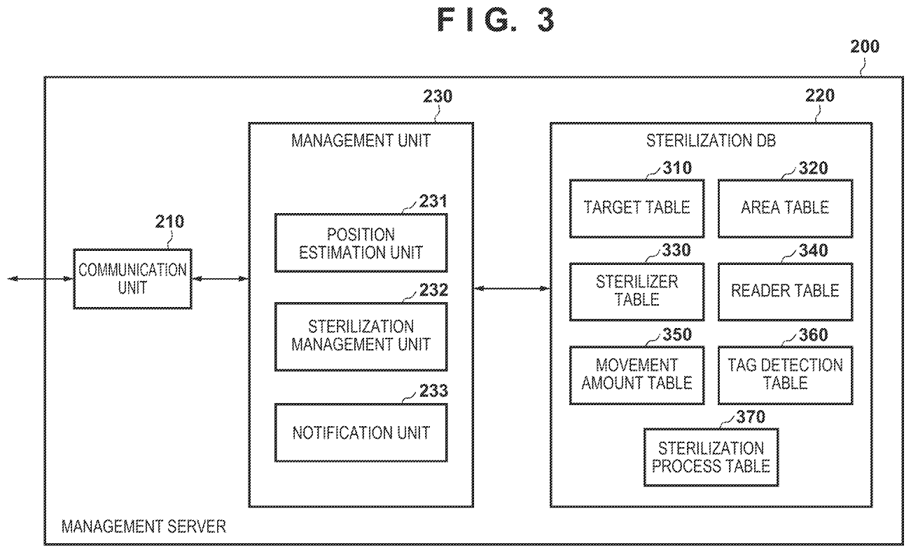

FIG. 3 is a block diagram illustrating an example of a configuration of the management server 200 according to an embodiment. With reference to FIG. 3, the management server 200 comprises a communication unit 210, a sterilization database (DB) 220, and a management unit 230.

The communication unit 210 is a communication interface for the management server 200 to communicate with other apparatuses. The communication unit 210 may be a wired communication interface or a wireless communication interface. In the present embodiment, the communication unit 210 communicates with the terminal apparatus 90 and tag readers 100. The sterilization DB 220 is a database that stores data regarding RFID tags and sterilization targets under management of the system. In the present embodiment, the sterilization DB 220 includes a target table 310, an area table 320, a sterilizer table 330, a reader table 340, a movement amount table 350, a tag detection table 360, and a sterilization process table 370. The management unit 230 is a set of software modules that provide management functions for managing data within the sterilization DB 220. The individual software modules can run by one or more processors (not shown) of the management server 200 executing computer programs stored in a memory (not shown). In the present embodiment, the management unit 230 includes a position estimation unit 231, a sterilization management unit 232, and a notification unit 233.

### <3-2. Data Configuration Examples>

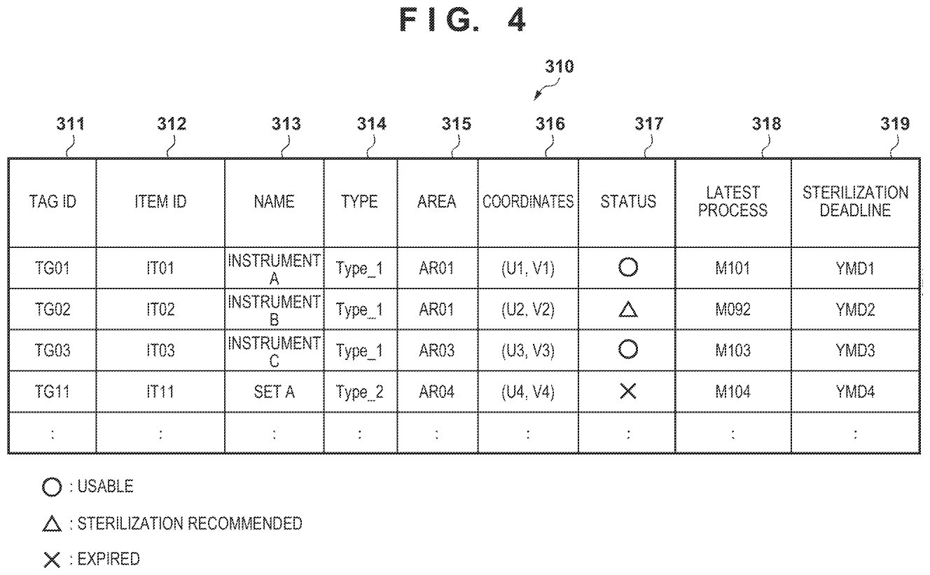

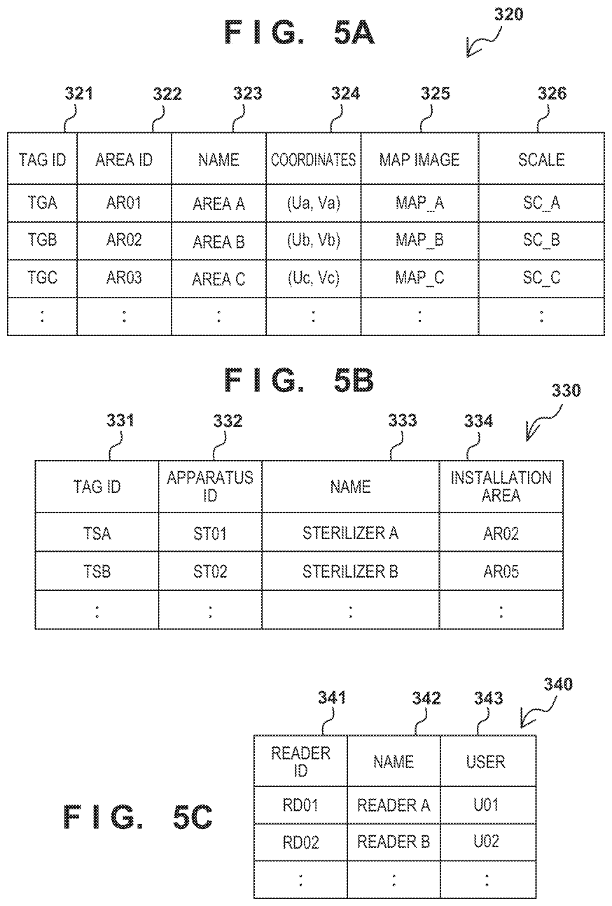

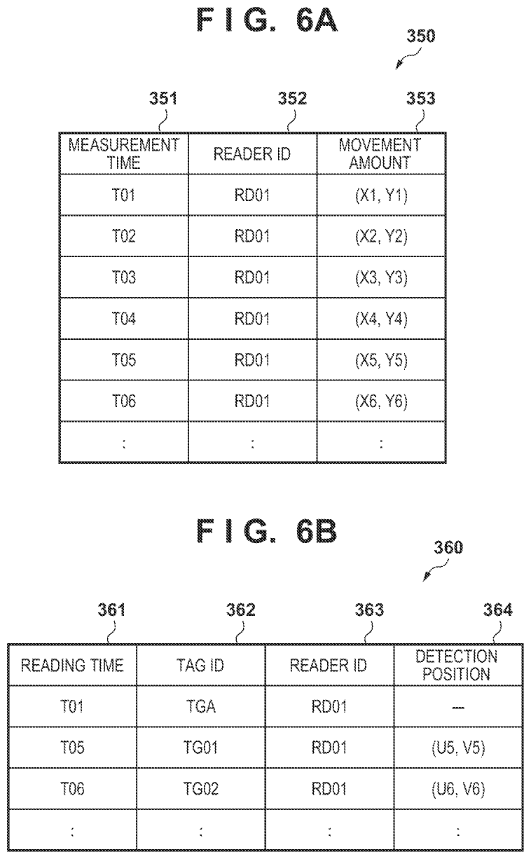

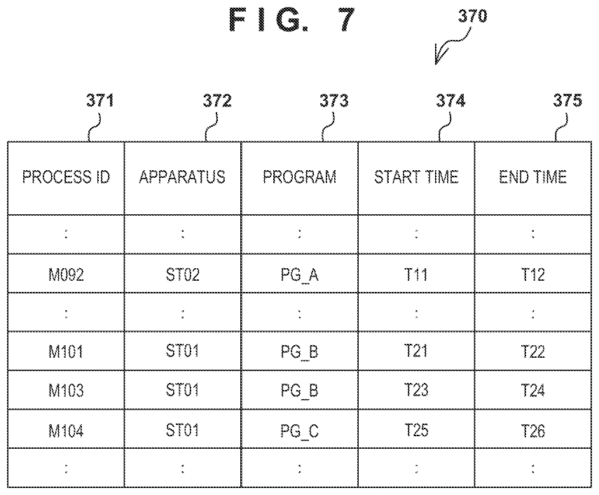

FIG. 4 illustrates an example of a configuration of the target table 310 of the sterilization DB 220. FIGS. 5A to 5C illustrate respective examples of configurations of the area table 320, the sterilizer table 330, and the reader table 340 of the sterilization DB 220. FIGS. 6A and 6B illustrate respective examples of configurations of the movement amount table 350 and the tag detection table 360. FIG. 7 illustrates an example of a configuration of the sterilization process table 370.

The target table 310 has nine data elements, namely Tag ID 311, Item ID 312, Name 313, Type 314, Area 315, Coordinates 316, Status 317, Latest Process 318, and Sterilization Deadline 319. Tag ID 311 is identification information uniquely identifying an item tag 50 attached to each of the items 30 that are sterilization targets. The value of Tag ID 311 is the same as the value of the tag ID stored within the corresponding item tag 50. Item ID 312 is identification information that uniquely identifies each item 30. Name 313 represents a name of each item 30. Type 314 represents a type into which each item 30 is classified. The values of Name 313 and Type 314 of each item 30 are determined by a user, and may be registered in advance via a user interface provided by the management unit 230. Alternatively, the values of Name 313 and Type 314 may be stored in item tags 50 as item-related information and read by a tag reader 100. In the latter case, upon initial tag reading from the item tag 50 of each item 30, the management server 200 may receive the values of Name 313 and Type 314 of that item 30 from the tag reader 100, and register them in the target table 310.

Area 315 represents an area in which each item 30 has been last detected by a tag reader 100 with an area ID identifying that area. Coordinates 316 represents positional coordinates of a point at which each item is estimated to be positioned. The values of Area 315 and Coordinates 316 are examples of the position information. Such position information may be updated by the position estimation unit 231 when a tag reader 100 has detected a movement of an item as will be described below.

Status 317 is status information representing a sterilization state for each sterilization target. In the example of FIG. 4, the statuses of sterilization targets identified by item IDs “IT01” and “IT03” indicate “Usable”. This means that there remain sufficient number of days until the upcoming deadline of sterilization process of each sterilization target and there is no problem to use the sterilization target. The status of a sterilization target identified by an item ID “IT02” indicates “Sterilization Recommended”. This means that there remain only few days until the upcoming deadline of sterilization process of the sterilization target and early execution of the sterilization process is recommended for the sterilization target. Both of these two kinds of status values represent that the sterilization target is still in a low-risk sterilization state. Meanwhile, the status of a sterilization target identified by an item ID “IT11” indicates “Expired”. This means that the deadline of sterilization process has already passed. This status value represents that the sterilization target is in a high-risk sterilization state, and it is not recommended or it is prohibited to use the sterilization target. Latest Process 318 represents a sterilization process that has been last performed for each sterilization target with a process ID identifying that sterilization process. Sterilization Deadline 319 represents a deadline by which a next sterilization process should be performed for each sterilization target. The deadline of sterilization process is, in principle, the date on which an effective period of sterilization which is calculated from a previous date of sterilization process expires. The effective period of sterilization may be preset depending on a type of the sterilization target and conditions of the sterilization process, for example. Sterilization Deadline 319 is an example of timing information representing a timing of the sterilization process to be performed for each sterilization target. The target table 310 may have data elements respectively representing a sterilization process date (the date on which a sterilization process was previously performed) and an effective period, instead of (or in addition to) Sterilization Deadline 319 as the timing information. Status 317, Latest Process 318, and Sterilization Deadline 319 are examples of sterilization-related information. Upon a sterilization process having been performed for each sterilization target, such sterilization-related information may be obtained by the sterilization management unit 232 and reflected in the target table 310 as described below.

The area table 320 has six data elements, namely Tag ID 321, Area ID 322, Name 323, Coordinates 324, Map Image 325, and Scale 326. Tag ID 321 is identification information that uniquely identifies a position tag 40 installed in each of the areas 10 under management of the system. The value of Tag ID 321 is the same as the value of the tag ID stored within the corresponding position tag 40. Area ID 322 is identification information that uniquely identifies each area 10. Name 323 represents a name of each area 10. In the example of FIG. 5A, the names of areas 10 identified by the area IDs “AR01”, “AR02”, and “AR03” are “Area A”, “Area B”, and “Area C”, respectively. Each name may be, in practice, such as “Work Site”, “Sterilization Room”, “Office”, “Entrance”, “Locker Room”, “1st Floor”, or “Separate Building”, for example. Coordinates 324 represents positional coordinates of an installation position of a position tag 40 installed in each area 10. Map Image 325 is a data element in which map image data of each area 10 is stored. Scale 326 indicates a ratio for converting a distance on the map of Map Image 325 into a distance in the real space (for example, how many meters in the real space one pixel of the image corresponds to). Note that the map image data stored in Map Image 325 may be updated with one obtained from an external data source or uploaded by a user at necessary timings.

The sterilizer table 330 has four data elements, namely Tag ID 331, Apparatus ID 332, Name 333, and Installation Area 334. Tag ID 331 is identification information that uniquely identifies a sterilization tag 80 installed on each of the sterilizers 60 under management of the system. The value of Tag ID 331 is the same as the value of the tag ID stored within the corresponding sterilization tag 80. Apparatus ID 332 is identification information that uniquely identifies each sterilizer 60. Name 333 represents a name of each sterilizer 60. Installation Area 334 represents an area in which each sterilizer 60 is installed with an area ID identifying that area.

The reader table 340 has three data elements, namely Reader ID 341, Name 342, and User 343. Reader ID 341 is identification information that uniquely identifies each of the tag readers 100 utilized in the system. Name 342 represents a name of each reader. User 343 identifies a user 20 who uses each tag reader 100 with a user ID identifying that user 20. The sterilization DB 220 may have an independent table that maintains user information of each of the users identified by user IDs (for example, username, organization, authentication information for use in login, and the like). Alternatively, the user information may be maintained by a separate database (for example, an authentication DB).

The movement amount table 350 is a table for accumulating records of measurement result data received from tag readers 100 (hereinafter referred to as measurement result records). The movement amount table 350 has three data elements, namely, Measurement Time 351, Reader ID 352, and Movement Amount 353. Measurement Time 351 indicates a time at which measurement was performed for the measurement result indicated by each measurement result record. Reader ID 352 is identification information that identifies a tag reader 100 that has performed the measurement for the measurement result indicated by each measurement result record. In the example of FIG. 6A, six records in the movement amount table 350 indicate the results of measurements of amounts of movement that have been performed by the tag reader 100, which is identified by Reader ID “RD01”, at six different times “T01” to “T06”. Movement Amount 353 represents a relative amount of movement as a measurement result. Herein, Movement Amount 353 represents the relative amount of movement in the form of a two-dimensional vector in a real-space coordinate system.

The tag detection table 360 is a table for accumulating records of reading result data received from the tag readers 100 (hereinafter referred to as reading result records). The tag detection table 360 has four data elements, namely Reading Time 361, Tag ID 362, Reader ID 363, and Detection Position 364. Reading Time 361 represents a time at which a tag ID was read for each reading result record. Tag ID 362 represents the tag ID that has been read for each reading result record. Reader ID 363 is identification information that identifies the tag reader 100 that has performed tag reading for each reading result record. In the example of FIG. 6B, the first record in the tag detection table 360 indicates that the tag reader 100a identified by the reader ID “RD01” has read the tag ID “TGA” (for example, the tag ID 41a of the position tag 40a in the area 10a) at time “T01”. The second record indicates that the tag reader 100a has read the tag ID “TG01” (for example, the tag ID 51a of the item tag 50a of item 30a) at time “T05”. The third record indicates that the tag reader 100a has read the tag ID “TG02” (for example, the tag ID 51b of the item tag 50b of item 30b) at time “T06”. Detection Position 364 represents positional coordinates of a point at which the tag reader 100 has existed at the time of tag reading from an item tag 50 (that is, the detection position of an item tag 50 and a corresponding item 30).

The sterilization process table 370 is a table for maintaining information regarding sterilization processes performed by each sterilizer 60. The sterilization table 370 has five data elements, namely Process ID 371, Apparatus 372, Program 373, Start Time 374, and End Time 375. Process ID 371 is identification information that uniquely identifies each sterilization process. Apparatus 372 represents a sterilizer 60 that performed each sterilization process with an apparatus ID identifying that sterilizer 60. Program 373 is identification information that identifies a set of process conditions set in the sterilizer 60 at the time of performing each sterilization process. The process conditions may include one or more of elements such as set temperature, process duration, and type of sterilization agent, for example. Start Time 374 represents a time at which each sterilization process started. End Time 375 represents a time at which each sterilization process ended.

### <3-3. Estimation of Position>

The position estimation unit 231 adds, to the movement amount table 350, each record of the measurement result data received from the tag readers 100 via the communication unit 210 as a measurement result record. In addition, the position estimation unit 231 adds, to the tag detection table 360, each record of the reading result data received from the tag readers 100 via the communication unit 210 as a reading result record. Then, the position estimation unit 231 estimates a current position of a tag reader 100 based on a relative amount of movement of the tag reader 100 from a point in time at which the tag reader 100 has read a tag ID from a position tag 40 and the known position of that position tag 40. In addition, in response to a tag ID having been read from an item tag 50 by the tag reader 100, the position estimation unit 231 estimates a position of the item 30 to which that item tag 50 is attached. The estimated position of the item 30 may be equal to the current position of the tag reader 100 at the time point when the item 30 has been detected, for example. The position estimation unit 231 adds the positional coordinates representing the estimated position of each item 30 to the field of Detection Position 364 of the tag detection table 360. In addition, the position estimation unit 231 determines to which area the latest estimated position of each item 30 belongs, and updates the values of Area 315 and Coordinates 316 of the target table 310 with the area ID of the determined area and positional coordinates representing the latest position.

For example, the relative amount of movement of the tag reader 100 at a first point in time when the tag ID is read from the position tag 40 by the tag reader 100 is defined as (X₀, Y₀), and positional coordinates of the known installation position of that position tag 40 is defined as (U₀, V₀). Also, the relative amount of movement of that tag reader 100 at a second point in time when the tag ID is read from an item tag 50 by the same tag reader 100 is defined as (X, Y). Accordingly, the position (U, V) of the sterilization target to which the item tag 50 is attached can be derived according to the following Formula (1):

$$( U , V ) = ( {U}_{0} + ( X - {X}_{0} ) , \qquad ( 1 )$$

$${V}_{0} + ( Y - {Y}_{0} ) )$$

Formula (1) is premised on estimating a two-dimensional position, but it can easily be expanded to estimate a three-dimensional position. As described above, the position estimation unit 231 may estimate that the sterilization target exists at the position derived according to Formula (1). Also, when the same item tag 50 is detected a plurality of times in a certain time period, the position estimation unit 231 may estimate that the corresponding sterilization target exists at the center (centroid position) of the plurality of estimated positions derived according to Formula (1). In the sterilization management system 1 according to the present embodiment, the position of each sterilization target is estimated in this manner, and the position information indicating the estimated positions is accumulated in the sterilization DB 220.

### <3-4. Obtaining Sterilization-Related Information

The sterilization management unit 232 obtains the sterilization-related information output by the sterilizer 60 when the sterilization process is performed by the sterilizer 60. The sterilization-related information output by the sterilizer 60 may include, for example, a process ID for identifying each sterilization process, an apparatus ID for identifying the sterilizer 60 which is the output source, program information, and information (for example, start time and end time) representing the process time period from the start to the end of the sterilization process. In a case where the sterilizer 60 has a communication function for communicating with the management server 200, the sterilization management unit 232 may receive the sterilization-related information from the sterilizer 60 via the communication unit 210. In a case where the sterilizer 60 has no communication function, the sterilization management unit 232 may obtain the sterilization-related information (uploaded or manually input in a data file format) via the I/O interface of the management server 200 or the terminal apparatus 90. The sterilization management unit 232 stores the obtained sterilization-related information in the sterilization process table 370.

In the present embodiment, the sterilization management unit 232 further associates the sterilization-related information stored in the sterilization process table 370 with the sterilization targets subjected to the sterilization process. Specifically, the sterilization management unit 232 determines temporal correlation between a third point in time when the tag ID has been read from the sterilization tag 80 by the tag reader 100 and a fourth point in time when the tag ID has been read from the item tag 50 by the same tag reader 100. For example, when the time difference between the third point in time and the fourth point in time is less than a preset threshold, it is presumed that the sterilization target corresponding to the corresponding sterilizer 60 being operated by the user 20 exists close by. Then, in a case where the third point in time and the fourth point in time are temporally close (for example, just before or after the processing period) to the processing period of the sterilization process performed by the sterilizer 60, there is a high possibility that the corresponding sterilization target has been sterilized in the sterilization process. The sterilization management unit 232, on the basis of this correlation, updates the values of the Status 317, the Latest Process 318, and the Sterilization Deadline 319 of the target table 310 corresponding to the sterilization target detected by the tag reader 100 with the new information.

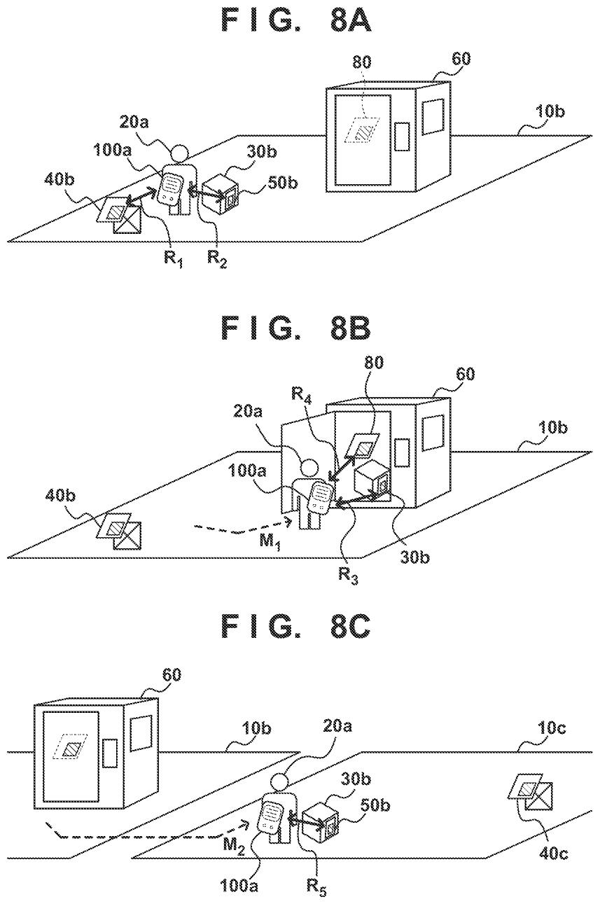

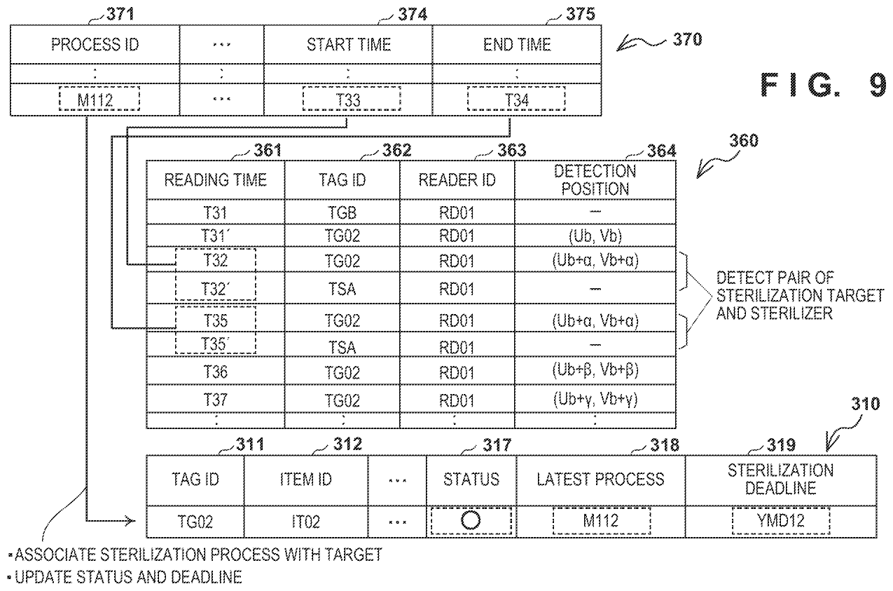

FIGS. 8A to 8C is an explanatory diagram for explaining an example of a scenario related to sterilization of a sterilization target in which a user is involved. FIG. 9 is an explanatory diagram for explaining association of a sterilization target with a sterilization process in the scenario of FIGS. 8A to 8C.

FIG. 8A illustrates the user 20a carrying the tag reader 100a bringing the item 30b, the sterilization target, into the area 10b. At time T31, the tag reader 100a reads (arrow R₁) the tag ID from the position tag 40b installed in the area 10b and transmits the reading result data and measurement result data to the management server 200. The first reading result record of the tag detection table 360 in the middle section of FIG. 9 is the record corresponding to this reading result data. Also, at time T31′, the tag reader 100a reads (arrow R₂) the tag ID from the item tag 50b of the item 30b the user 20a is carrying and transmits the reading result data and the measurement result data to the management server 200. The position estimation unit 231 of the management server 200 estimates the latest position of the item 30b on the basis of the reading result data and the measurement result data received from the tag reader 100a. The second reading result record of the tag detection table 360 in the middle section of FIG. 9 is the record corresponding to this reading result data, with the estimated position of the item 30b at this point in time being represented by the value in Detection Position 364.

FIG. 8B illustrates, in the area 10b, the user 20a carrying the tag reader 100a putting the item 30b into the sterilizer 60 after moving according to a dashed line arrow M₁. At time T32 during the work of the user 20a, the tag reader 100a re-reads (arrow R₃) the tag ID from the item tag 50b of the item 30b and transmits the reading result data and the measurement result data to the management server 200. The position estimation unit 231 of the management server 200 estimates the latest position of the item 30b on the basis of the reading result data and the measurement result data received from the tag reader 100a. The third reading result record of the tag detection table 360 in the middle section of FIG. 9 is the record corresponding to this reading result data. Then, at time T32′ close to time T32, the tag reader 100a reads (arrow R₄) the tag ID from the sterilization tag 80 installed inside the sterilizer 60 and transmits the reading result data to the management server 200. The fourth reading result record of the tag detection table 360 in the middle section of FIG. 9 is the record corresponding to this reading result data.

Thereafter, the sterilizer 60 performs the sterilization process on the item 30b and outputs the sterilization-related information relating to the performed sterilization process to the management server 200. The sterilization process table 370 in the top section of FIG. 9 is a partial representation of the sterilization-related information output to the management server 200 at this time. For example, of the performed sterilization process, the Process ID is “M112”, the Start Time is T33, and the End Time is T34.

Though not illustrated in FIGS. 8A to 8C, after the sterilization process ends, at time T35 and time T35′ while the user 20a is removing the item 30b from the sterilizer 60, the tag reader 100a reads the tag ID from the item tag 50b and the sterilization tag 80. The fifth and sixth reading result record of the tag detection table 360 in the middle section of FIG. 9 illustrates the reading results of these tag IDs.

FIG. 8C illustrates the user 20a moving the item 30b after the sterilization process from the area 10b to the area 10c according to a dashed line arrow M₂. While the user 20a is carrying the item 30b, over a plurality of times, the tag reader 100a reads (arrow R₅) the tag ID from the item tag 50a and transmits the reading result data and the measurement result data to the management server 200 a number of times. The position estimation unit 231 of the management server 200 tracks the position of the item 30b. The seventh and eighth reading result record of the tag detection table 360 in the middle section of FIG. 9 illustrate the estimated positions of the item 30b continuously tracked represented by the values in Detection Position 364.

The sterilization management unit 232, from the reading result record of the tag detection table 360 illustrated in FIG. 9, recognizes that the item tag 50b of the item 30b has been detected at time T32 which is roughly the same time as time T32′ when the sterilization tag 80 was detected. In a similar manner, the sterilization management unit 232 recognizes that the item tag 50b of the item 30b has been detected at time T35 which is roughly the same time as time T35′ when the sterilization tag 80 was detected. Also, the sterilization management unit 232 references the sterilization process table 370 and recognizes that the sterilization process identified by Process ID “M112” was performed by the sterilizer 60 from time T33 to time T34. For example, time T32 is just before T33, and time T35 is just after time T34. On the basis of this knowledge, the sterilization management unit 232 determines that the sterilization process identified by Process ID “M112” has been performed on the item 30b and associated the sterilization-related information of this sterilization process with the item 30b. In the example in the lower section of FIG. 9, the value of Latest Process 318 for the record corresponding to the item 30b of the target table 310 has been updated to “M112”. Also, the value of Sterilization Deadline 319 for this record has been updated to the next sterilization process deadline (for example, the date on which a predetermined effective period calculated from the process date of the latest sterilization process expires). The value of Status 317 may also be updated.

In an example, the sterilization management unit 232 may associate the sterilization-related information of the sterilization process with the item 30 on the condition that a pair of the item tag 50 and the sterilization tag 80 have been detected at both before and after the processing period and not just one of before and after the processing period of the sterilization process. Confirming the detection of a pair of tags at both before and after the processing period can reduce the possibility of erroneously associating a sterilization process with a sterilization target in a case where a sterilization target that has been put in the sterilizer is removed before the sterilization process is performed. In another example, a particular user operation (for example, a button press) at the operation unit 115 of the tag reader 100 may be predefined for associating a sterilization target with a sterilization process. In this case, the control unit 111 of the tag reader 100 provides a flag indicating detection of the user operation to the reading result data indicating the result of tag reading while the user operation is detected and transmits this to the management server 200. When the sterilization management unit 232 receives the reading result data including the tag ID of the item tag 50 and this flag, the sterilization management unit 232 determines that the sterilization process has been performed for the corresponding item 30 and associates the sterilization process performed at the corresponding time with the item 30. In this case, association can be more accurately determined on the basis of only the detection of a single pair of the item tag 50 and the sterilization tag 80.

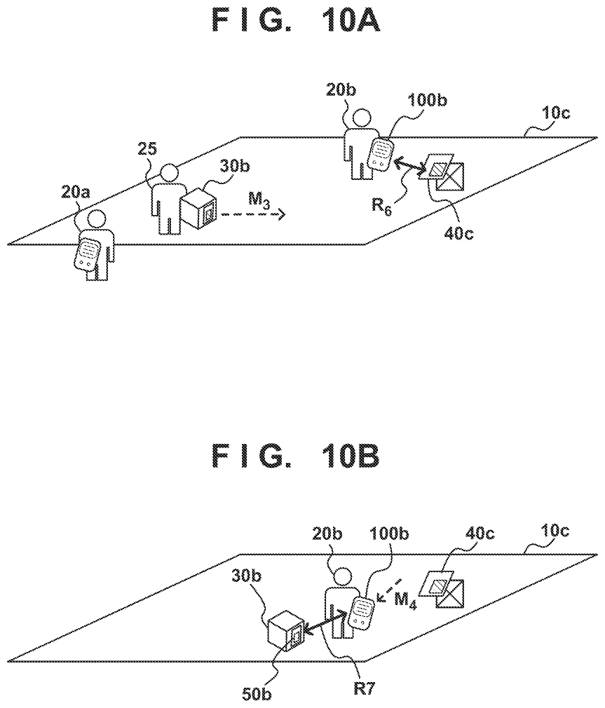

FIGS. 10A and 10B are explanatory diagram for explaining movement and detection of the item 30b, which is the sterilization target, following on from the scenario of FIGS. 8A to 8C. FIG. 10A illustrates, after the user 20a carries the item 30b into the area 10c, a person 25 without the tag reader 100 moving the item 30b according to dashed line arrow M₃. When the sterilization target is moved in this manner with various people being involved and due to various tasks, there is a possibility that the position tracking may be interrupted. However, in the present embodiment, with the sterilization management system 1, the plurality of tag readers 100 are each carried and operated by various users 20. For example, FIG. 10A illustrates the user 20b carrying the tag reader 100b visiting the area 10c. The tag reader 100b reads (arrow R₆) the tag ID from the position tag 40c installed in the area 10c. Also, as illustrated in FIG. 10B, when the user 20b moves according to dashed line arrow M₄ and approaches the item 30b, the tag reader 100b reads (arrow R₇) the tag ID from the item tag 50b of the item 30b. The user 20b may not intentionally approach the item 30b at this time. The tag reader 100b transmits the reading result data indicating the results of tag reading for the item 30b and the measurement result data to the management server 200. The position estimation unit 231 of the management server 200 estimates the latest position of the item 30b on the basis of the data received from the tag reader 100b and updates the position information in the sterilization DB 220. In this manner, the position of each sterilization target under the management of the sterilization management system 1 is tracked over time, and the position information is maintained in the sterilization DB 220.

Note that in this example, the association between the sterilization target and the sterilization process is performed on the basis of the detection of the sterilization tag 80 installed on the sterilizer 60. However, the association method is not limited to this example. Other methods for associating the sterilization target with the sterilization process will be further described below.

<3-5. User Notification for Assisting with Sterilization Management>

The notification unit 233, to assist with sterilization management, notifies the user 20 of the position where each sterilization target is estimated to exist on the basis of the position information stored in the sterilization DB 220 via a user interface provided by the terminal apparatus 90. The sterilization targets to be notified of may be selected on the basis of the above-described sterilization-related information stored in the sterilization DB 220. In this example, the user notification is mainly performed using a graphical user interface (GUI). However, the user notification may be performed using an audio interface or another type of UI.

The notification unit 233 accepts a user input designating a notification condition for selecting a sterilization target on the screen displayed on a display, for example. This notification condition may be a condition relating to the status information from among the sterilization-related information stored in the target table 310. In an example, the user 20 requests the position information of an appropriately sterilized sterilization target from the management server 200 in order to learn the location of a sterilization target that can be used while avoiding the risk of infection. The notification unit 233, in response to this request, selects one or more sterilization targets with “Usable” as the value for Status 317 in the target table 310 and notifies the user 20 of the estimated position of the selected sterilization target/s. In another example, the user 20 requests the position information of a sterilization target with an expired sterilization deadline from the management server 200 in order to find sterilization targets that promptly need to be sterilized. The notification unit 233, in response to this request, selects one or more sterilization targets with “Deadline Expired” as the value for Status 317 in the target table 310 and notifies the user 20 of the estimated position of the selected sterilization target/s.

Another notification condition may be a condition relating to the timing information from among the sterilization-related information stored in the target table 310. In an example, the user 20 requests the position information of a sterilization target with a low number of remaining days until the sterilization deadline from the management server 200 in order to sterilize the sterilization target in a timely manner. The notification unit 233, in response to this request, selects one or more sterilization targets with the number of remaining days until the date indicated by Sterilization Deadline 319 being equal to or less than a threshold in the target table 310 and notifies the user 20 of the estimated position of the selected sterilization target/s. The threshold compared here to the number of remaining days until sterilization deadline may be preset or variably designated by the user 20. The threshold may be any one of a positive number, zero (in other words, the current day being the deadline), or a negative number (in other words, the deadline is a date in the past).

In the present embodiment, the notification unit 233 obtains map image data of the area (for example, the area indicated by Area 315 of the target table 310) where the sterilization target exists from the area table 320 and displays the map image on the screen of the terminal apparatus 90. Then, by plotting the estimated positions of the one or more sterilization targets selected according to the above-described notification condition on the map image, the notification unit 233 notifies the user 20 of the estimated positions of these sterilization targets. By viewing the displayed map image, the user 20 can quickly and intuitively learn where a intended sterilization target exists.

For sterilization targets of which sterilization deadlines do not satisfy the designated notification condition, the notification unit 233 may not notify the user 20 of the positions where the sterilization targets are estimated to exist or may perform notification to the user 20 in a first notification mode. In the case of the latter, for sterilization targets of which sterilization deadlines satisfy the designated notification condition, the notification unit 233 notifies the user 20 of the positions where the sterilization targets are estimated to exist in a second notification mode more emphasized than the first notification mode. This makes it easier for the user 20 to promptly distinguish and find a sterilization target that needs to be sterilized from among multiple sterilization targets that may exist. Also, for a sterilization target of which sterilization deadline is a date in the past, the notification unit 233 may notify the user 20 of the position where the sterilization target is estimated to exist in a notification mode that is different from that of the other sterilization targets. This can suppress the possibility of the user 20 erroneously using a sterilization target with an expired effective period of sterilization and with hygiene concerns. Note that making the notification mode more emphasized (or changing the notification mode) may be performed by any method including, for example, changing the display color, changing the display size, flashing, or adding additional graphic elements.

In a case where the user 20 views the position information of a sterilization target while carrying the tag reader 100 and the terminal apparatus 90, the notification unit 233 may further plot the current position of the tag reader 100 estimated by the position estimation unit 231 on the above-described map image. In this case, the user 20 can find the desired sterilization target in a short amount of time while checking the positional relationship between the current position of the tag reader 100 they are carrying and the estimated position of the sterilization target. This also applies to cases where the tag reader 100 includes a user interface with a display and the position information is viewed using the tag reader 100. Note that in a case where the tag reader 100 and the portable terminal 90 are both carried by the user 20, the tag reader 100 and the portable terminal 90 are collectively referred to as a portable system.

The position information of the sterilization target notified by the notification unit 233 may only indicate the latest estimated position of the sterilization target or may indicate a plurality of estimated positions of the sterilization target corresponding to a plurality of points in time. In the case of the latter, the notification unit 233 may display the history of the estimated positions of the sterilization target across multiple points in time on the screen. Alternatively, the notification unit 233 may display the trajectory of the estimated positions of a time series of the sterilization target across multiple points in time on the screen. In a case where the user 20 cannot find the sterilization target at the latest estimated position, by referencing the position history or movement trajectory, the actual position can be deduced and the sterilization target can be efficiently found. Instead of notifying of the estimated position directly indicated by the position information, the notification unit 233 may predict the current position of the sterilization target from a plurality of previous estimated positions indicated by the position information and may notify the user 20 of the predicted current position of the sterilization target as the estimated position of the sterilization target. For example, the notification unit 233 may predict the current position of the sterilization target on the basis of cyclicity in the changes in position of the sterilization target or may predict that the current position of the sterilization target is in a place where the sterilization target frequently existed in the past.

In the present embodiment, the position information of the estimated positions of the sterilization targets corresponding a plurality of points in time accumulated in the tag detection table 360 is based on the reading result data and the measurement result data received from the plurality of tag readers 100 carried by different users 20. In the sterilization management system 1, with a greater number of operational tag readers 100, the possibility of detecting each of the sterilization targets existing in various places using the tag readers 100 is further increased. Alternatively, with increased activity of the users 20 moving the tag readers 100 or other mobile objects, the possibility of each sterilization target being detected by the tag readers 100 is increased. If the number of tag readers 100 and the amount of activities is sufficient, the sterilization targets will be detected by the tag readers 100 no matter where they exist. This strengthens the sterilization target detection capability of the overall system.

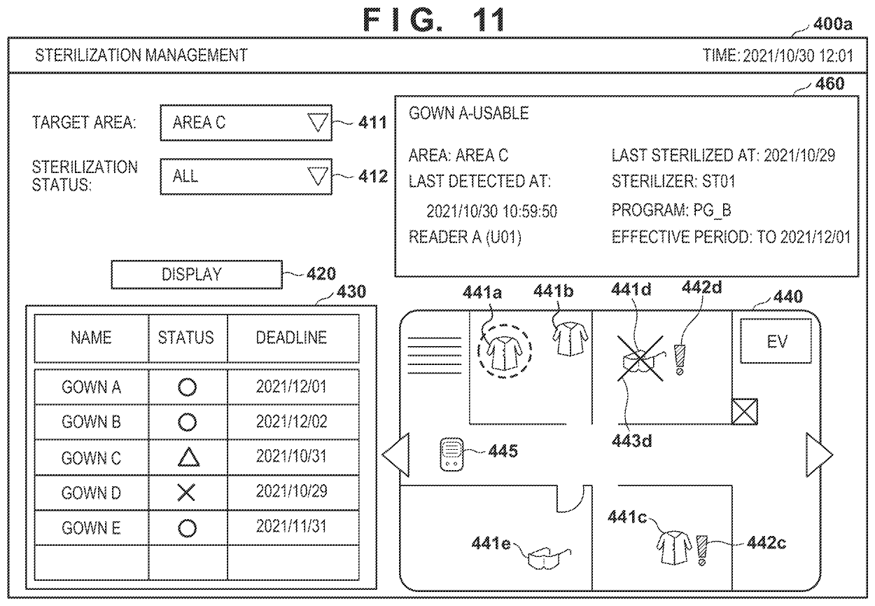

FIGS. 11 to 15 are explanatory diagrams for explaining examples of GUIs for assisting the user 20 with sterilization management. FIG. 11 illustrates a sterilization management screen 400a as an example. The sterilization management screen 400a includes an area designation field 411, a status condition field 412, a display button 420, a list information region 430, a map display region 440, and a detailed information region 460.

The area designation field 411 is a field (for example, a pull down menu) for allowing the user 20 to designate the area 10 to be displayed as a map image in the map display region 440. The status condition field 412 is a field (for example, a pull down menu) for allowing the user 20 to designate a status of sterilization targets to be plotted for estimated positions on the map image. For example, as illustrated in FIG. 11, the user 20 designates “Area C” in the area designation field 411 and “All” for the status condition field 412 and operates the display button 420. Then, the notification unit 233 selects, from among the sterilization targets registered in the target table 310, all of the sterilization targets estimated to exist in the area C according to the latest estimation results and displays a list of the selected sterilization targets in the list information region 430. In the example of FIG. 11, information such as the name, status, and sterilization deadline of each sterilization target is displayed in the list information region 430. However, another combination of any information may be displayed in the list information region 430.

The notification unit 233 further displays a map image of the area 10 designated by the user on the map display region 440 and plots the estimated positions of the select sterilization targets on the map image according to the designated condition. In the example of FIG. 11, in the map display region 440, five icons 441a to 441e representing five sterilization targets are plotted in the map image of the area C. The icon image of each icon 441 may be associated with the type of the corresponding sterilization target, for example. In the example of FIG. 11, the icon image for the icons 441a to 441c and the icon image for the icons 441d and 441e are different. Also, additional graphic elements 442c and 442d are added to the icon 441c and the icon 441d. The graphic elements 442c and 442d represent that the sterilization process needs to be performed promptly for the sterilization targets due to the number of remaining days until the sterilization deadline of the sterilization targets corresponding to the icon 441c and the icon 441d being equal to or less than the preset threshold. An additional graphic element 443d is added to the icon 441d. The graphic element 443d represents that use of the sterilization target is prohibited due to the sterilization deadline of the sterilization target corresponding to the icon 441d being a date in the past. In the example of FIG. 11, an icon 445 is further superimposed on the map image in the map display region 440. The icon 445, for example, represents the estimated current position of the tag reader 100 in a case where the sterilization management screen 400a is displayed on the terminal apparatus 90 carried by the user 20 together with the tag reader 100.

In the map display region 440, when the user 20 operates (for example, clicks or taps) one of the icons 441a to 441e, the notification unit 233 displays the detailed information on the sterilization target corresponding to the operated icon in the detailed information region 460. In the example of FIG. 11, as a result of the icon 441a (surround with a dashed line) being operated, the name, status, position information, and sterilization-related information of the sterilization target corresponding to the icon 441a are displayed on the detailed information region 460.

Note that the sterilization management screen 400a illustrated in FIG. 11 is merely an example of a screen for notifying the user 20 of the estimated positions of the sterilization targets. One or more of the illustrated components may be omitted from the screen, the layout of the components may be changed, or other components may be incorporated into the screen.

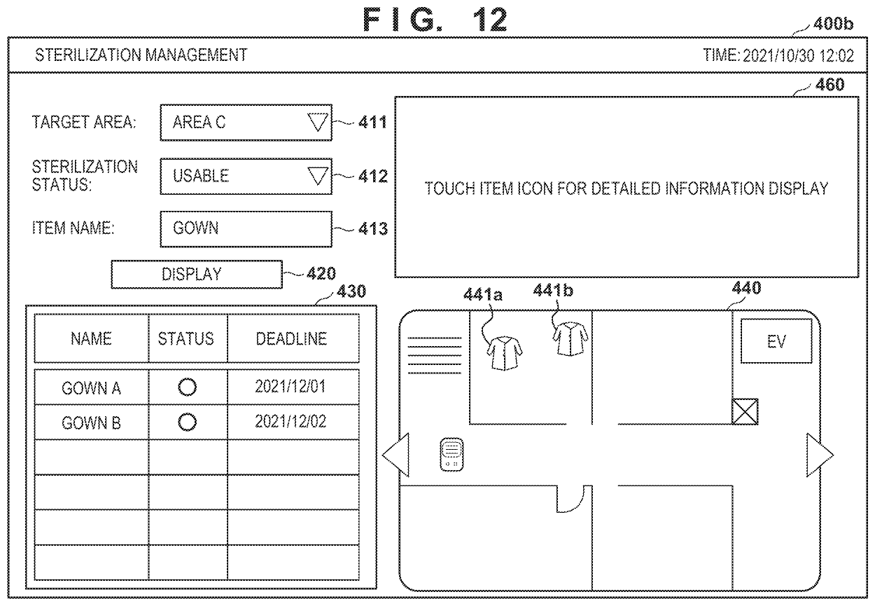

FIG. 12 illustrates a sterilization management screen 400b as an example. The configuration of the sterilization management screen 400b is substantially the same as the configuration of the sterilization management screen 400a illustrated in FIG. 11 except that the sterilization management screen 400b additionally includes an item name field 413. The item name field 413 is a text field for allowing the user 20 to designate (at least a part of) a name of a sterilization target to be plotted for estimated position on the map image. In the example of FIG. 12, “Area C” is designated in the area designation field 411, “Usable” is designated in the status condition field 412, and “Gown” is designated in the item name field 413. When the user 20 operates the display button 420 in this state, the notification unit 233 selects, from among the sterilization targets estimated to exist in the area C, sterilization targets with a sterilization status of “Usable” and the character string “Gown” in their names. Then, a list of the selected sterilization targets is displayed in the list information region 430 by the notification unit 233 and icons corresponding to the selected sterilization targets are plotted on the map image of the area C in the map display region 440. In the example of FIG. 12, the information of two sterilization targets with the names “Gown A” and “Gown B” is displayed in the list information region 430, and the two icons 441a and 441b corresponding to these sterilization targets are displayed in the map display region 440.

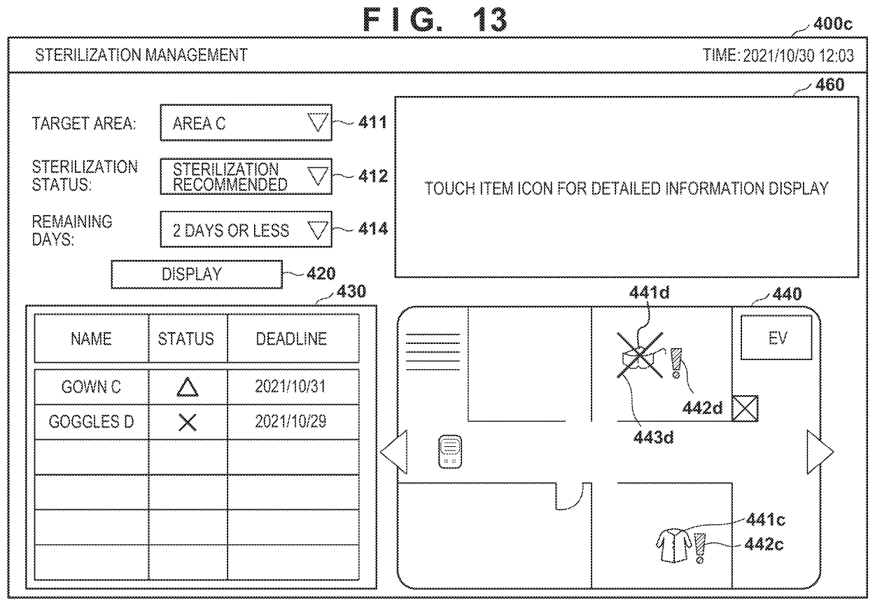

FIG. 13 illustrates a sterilization management screen 400c as an example. The configuration of the sterilization management screen 400c is substantially the same as the configuration of the sterilization management screen 400a illustrated in FIG. 11 except that the sterilization management screen 400c additionally includes a remaining days field 414. The remaining days field 414 is a field (for example, a pull down menu) for allowing the user 20 to designate a threshold for the number of remaining days until the sterilization deadline. In the example of FIG. 13, “Area C” is designated in the area designation field 411, “Needs Sterilization” is designated in the status condition field 412, and “Within Two Days” is designated in the remaining days field 414. When the user 20 operates the display button 420 in this state, the notification unit 233 selects, from among the sterilization targets estimated to exist in the area C, sterilization targets with a sterilization status of “needs sterilization” or “deadline expired” and the number of remaining days until the sterilization deadline being two days or less. Then, a list of the selected sterilization targets is displayed in the list information region 430 by the notification unit 233 and icons corresponding to the selected sterilization targets are plotted on the map image of the area C in the map display region 440. In the example of FIG. 13, the information of two sterilization targets with the names “Gown C” and “Goggles D” is displayed in the list information region 430, and the two icons 441c and 441d corresponding to these sterilization targets are displayed in the map display region 440. Also, the graphic element 442c representing that the sterilization process needs to be performed promptly is added to the icon 441c. The graphic element 442d also representing that the sterilization process needs to be performed promptly and the graphic element 443d representing that use is prohibited due to the sterilization effective period being expired are added to the icon 441d.

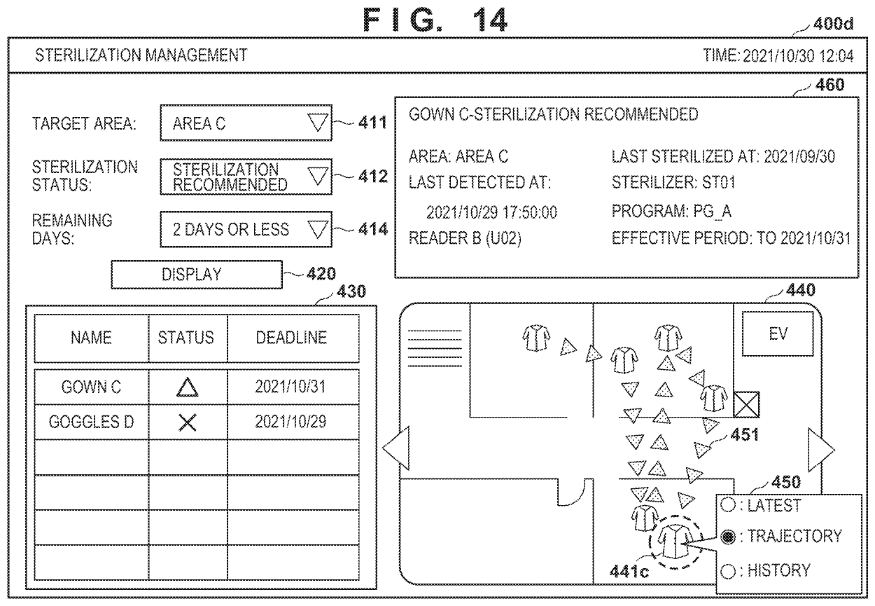

FIG. 14 illustrates a sterilization management screen 400d as an example. The configuration of the sterilization management screen 400d is similar to the configuration of the sterilization management screen 400c illustrated in FIG. 13. However, in the example of FIG. 14, a pop up menu 450 is displayed as a result of the user 20 operating the icon 441c. The pop up menu 450 is a menu that enables switching between three modes for the display mode of the estimated position of the selected sterilization target. The default display mode may be the display mode “Latest”, and in the “Latest” display mode, as illustrated in FIGS. 11 to 13, in the map display region 440, only the icons indicating the latest estimated position of each sterilization target are superimposed on the map image. In the example of FIG. 14, the display mode “Trajectory” is designated. In this case, the trajectory of the estimated positions of a time series of the previous N days (N>0) for the selected sterilization target is displayed by the notification unit 233 on the map display region 440 using graphic elements 451.

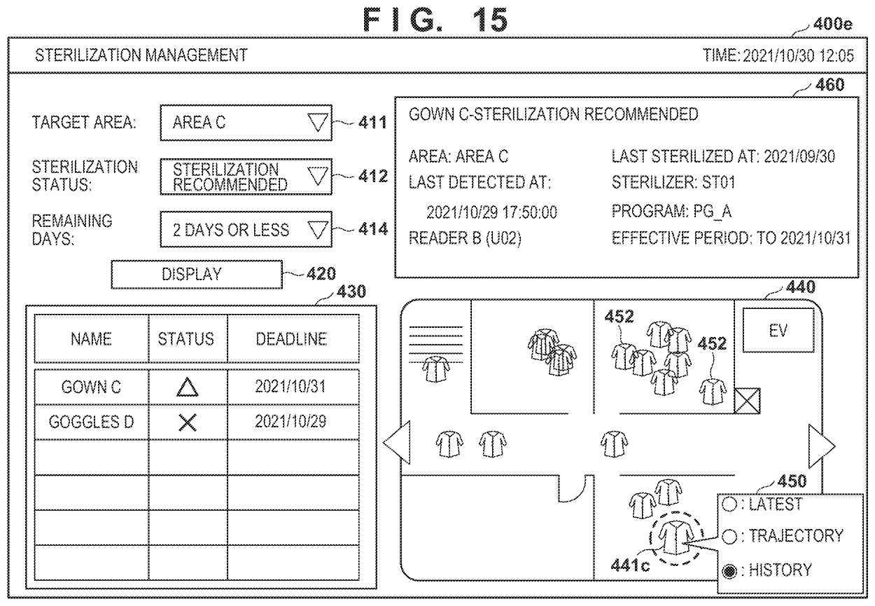

FIG. 15 illustrates a sterilization management screen 400e as an example. The configuration of the sterilization management screen 400e is similar to the configuration of the sterilization management screen 400d illustrated in FIG. 14. However, in the example of FIG. 15, in the pop up menu 450, the display mode “History” is designated. In this case, the history of the estimated positions over a plurality of times during the previous M days (M>0) for the selected sterilization target is displayed by the notification unit 233 on the map display region 440 using graphic elements 452. In the “History” display mode, compared to the “Trajectory” display mode, instead of the trajectory of the positions of a time series being represented on the screen, more estimated positions (over a longer time period) can be indicated to the user 20 on the screen. The values of the time period parameters N and M may be set to any value by the user 20.

In the example described relating to FIGS. 11 to 15, various conditions for selecting sterilization targets of which estimated positions are to be notified are used. However, naturally, conditions other than those described here may be used. For example, conditions relating to the number of times the sterilization process has been performed previously, the sterilization process date, or the sterilizer used for the sterilization process may be used.

### 4. Configuration Example of Sterilizer

### <4.1. Basic Configuration Example>

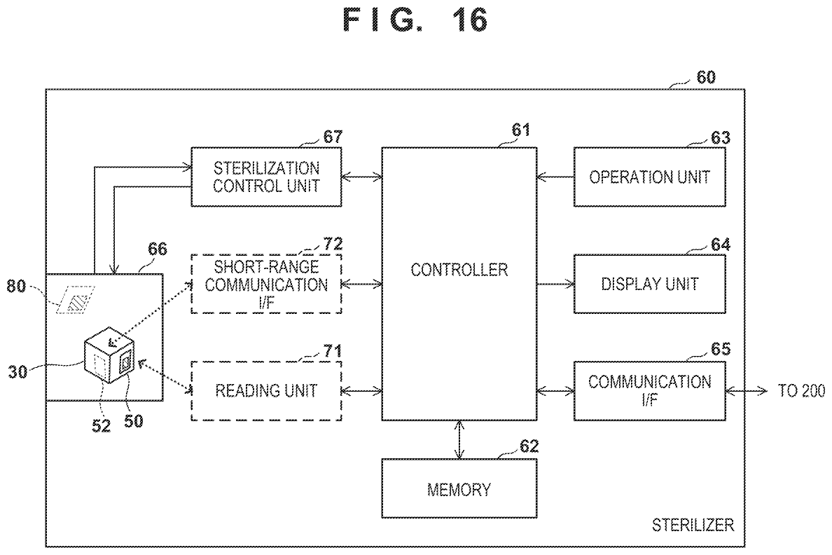

FIG. 16 is a block diagram illustrating an example of the sterilizer 60 according to an embodiment. Referencing FIG. 16, the sterilizer 60 includes a controller 61, a memory 62, an operation unit 63, a display unit 64, a communication I/F 65, a sterilization chamber 66, and a sterilization control unit 67.

The controller 61 is a control circuit that controls overall functionality of the sterilizer 60. The memory 62 stores one or more computer programs relating to the sterilization process and stores various parameters and data. The controller 61 may include a processor that executes the computer programs stored in the memory 62. The operation unit 63 is a unit for accepting operations by the user 20. The operation unit 115 may include one or more of a button, a switch, a touch panel, and a keyboard, for example. The display unit 64 is a unit for displaying information and images to the user 20. The communication I/F 65 is an interface that mediates communication with the management server 200 by the sterilizer 60.

The sterilization chamber 66 is a chamber with an internal space that houses the items 30 (in other words, the sterilization targets) to be subjected to the sterilization process. The sterilization chamber 66 is provided with an openable/closable door (not illustrated). For example, with the door of the sterilization chamber 66 in an open state, the user 20 can put the items 30 in the sterilization chamber 66 and can remove the items 30 from the sterilization chamber 66. The sterilization process is performed with the door of the sterilization chamber 66 in a closed state. As described above, in the present embodiment, the sterilization tag 80 is installed inside the sterilization chamber 66. The sterilization control unit 67 is a control circuit that controls performing the sterilization process on the items 30 inside the sterilization chamber 66. The sterilization control unit 67 exposes the items 30 inside the sterilization chamber 66 to high pressure steam or a sterilization agent or irradiates the items 30 inside the sterilization chamber 66 with ultraviolet light in accordance with the process conditions instructed by the controller 61, for example. The sterilizer 60 is actually provided with many components such as a sensor, an actuator, and the like for performing the sterilization process, but these components are omitted from FIG. 16 for the sake of simplifying the description. When execution of the sterilization process has finished, the controller 61 generates sterilization-related information that may include the apparatus ID of the sterilizer 60, program information, and information indicating the processing period of the sterilization process and outputs the generated sterilization-related information to the management server 200 via the communication I/F 65. Each sterilization process is provided with a process ID for identifying the sterilization process.

### <4.2. Some Alteration Examples>

In an alteration example, the sterilizer 60 may further include a reading unit 71. The reading unit 71 has the same function as the reading unit 116 of the tag reader 100 described above and can read the tag ID from the item tag 50 attached to the item 30. In this alteration example, the sterilization tag 80 may not be installed inside the sterilization chamber 66. When the sterilization process is performed, the controller 61 attempts to cause the reading unit 71 to perform tag reading form the item tag 50 of the item 30 put into the sterilization chamber 66 and obtains the read tag ID from the reading unit 71. Then, when the sterilization process ends, the controller 61 outputs the obtained tag ID and the sterilization-related information to the management server 200. According to such a configuration, the sterilization management unit 232 of the management server 200 can associate the appropriate sterilization target with each sterilization process on the basis of the tag ID output from the sterilizer 60 without relying on the reading result of the tag ID from the sterilization tag 80.

In another alteration example, the sterilizer 60 may further include a short-range communication I/F 72. The short-range communication I/F 72 may be a communication I/F that uses, for example, Bluetooth Low Energy (BLE), ZigBee, or any type of wireless communication protocol that can operate at low power. In this alteration example, a memory device 52 is further attached to the item 30. The memory device 52 operates based on the wireless communication protocol used by the short-range communication I/F 72 and can write data to the memory device 52 and read out data from the memory device 52. For example, when the sterilization process on the item 30 ends, the controller 61 writes information such as the sterilization process date and the sterilization effective period to the memory device 52 via the short-range communication I/F 72. Then, even if the item 30 is moved after being removed from the sterilizer 60, another apparatus that can communicate in accordance with the same wireless communication protocol can be used to read out the information from the memory device 52, and the user 20 can determine whether the sterilization of the item 30 is still effective. Also, in the above-described embodiment, the sterilization-related information is output from the sterilizer 60 to the management server 200 via the communication I/F 65. However, the sterilization-related information may be transmitted to the management server 200 via the memory device 52 (and another apparatus as necessary).

The information written to the memory device 52 may include program information of the sterilization process performed on the item 30. In this case, the controller 61 of the sterilizer 60 may read out the program information of the previous sterilization process from the memory device 52 of the item 30 put in the sterilization chamber 66 via the short-range communication I/F 72, and this may be reused for setting the process conditions of the next sterilization process.

The memory device 52 may further include a simple display (for example, a small liquid crystal panel, a seven-segment display, or the like), and the information such as the effective period written to the memory device 52 may be displayed on this display. Accordingly, the user 20 can determine whether the sterilization of the item 30 is effective by simply looking at the display attached to the item 30.

### 5. Process Flow

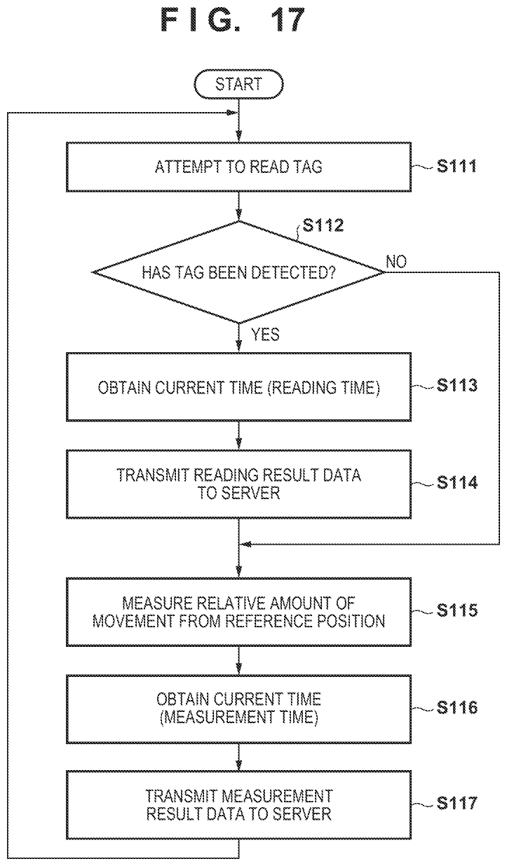

In the present segment, an example of the flow of some processes that may be performed in the sterilization management system 1 will be described using the flowcharts of FIGS. 17 to 20. Note that in the following description, process step is shortened to S (step).

### <5-1. Data Transmission Process (Tag Reader)>

FIG. 17 is a flowchart illustrating an example of the flow of a data transmission process performed by the tag reader 100. The data transmission process of FIG. 17 may be repetitively performed while the tag reader 100 is operating.

First, in S111, the reading unit 116 emits electromagnetic waves in a reading range to attempt to read a tag ID from nearby RFID tags. When the tag reading attempt results in a tag ID being received from a nearby RFID tag utilizing energy of electromagnetic waves (Yes in S112), the process proceeds to S113. On the other hand, when no tag ID is received (No in S112), the process proceeds to S115.

In S113, the control unit 111 references the internal real-time clock, for example, to obtain the current time as the reading time for the tag ID. Next, in S114, the control unit 111 transmits the reading result data including the read tag ID, the reading time, and the reader ID of the tag reader 100 to the management server 200 via the communication unit 113. Then, the process proceeds to S115.

In S115, the measuring unit 114 measures the relative amount of movement from a reference position on the basis of sensor data output from the three-axis acceleration sensor, the gyro sensor, and the geomagnetic sensor, for example. Next, in S116, the control unit 111 obtains the current time as the measurement time. Then, in S117, the control unit 111 transmits the measurement result data including the relative amount of movement measured by the measuring unit 114, the measurement time, and the reader ID of the tag reader 100 to the management server 200 via the communication unit 113. Then, the process returns to S111.

### <5-2. Data Update Process (Management Server)>

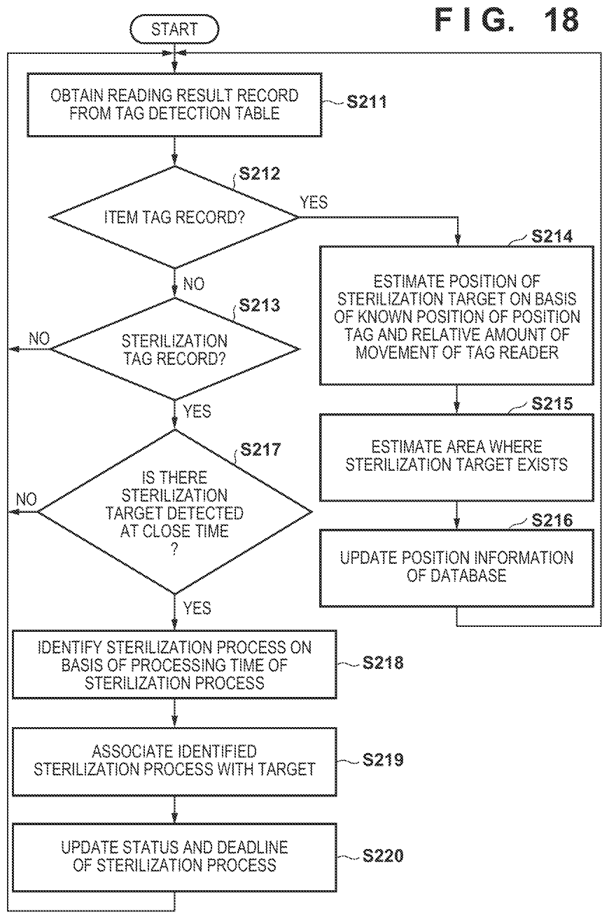

FIG. 18 is a flowchart illustrating an example of the flow of the data update process performed by the management server 200. The data update process of FIG. 18 may be performed each time a reading result record is added to the tag detection table 360 or may be periodically performed every couple of hours or once per day, for example. Though omitted from the flowchart, in the movement amount table 350 and the tag detection table 360 of the sterilization DB 220, records of the measurement result data and the reading result data received from the tag readers 100 are sequentially accumulated.

First, in S211, the position estimation unit 231 of the management server 200 obtains one newly added reading result record from the tag detection table 360. The process from then on branches at S212 and S213 depending on what type of RFID tag is indicated by the tag ID in the obtained reading result record. In a case where the obtained reading result record indicates a tag ID of an item tag 50 (Yes in S212), the process proceeds to S214. Also, in a case where the obtained reading result record indicates a tag ID of a sterilization tag 80 (Yes in S213), the process proceeds to S217. In a case where the obtained reading result record indicates a tag ID of a position tag 40, the process returns to S211, and the next reading result record from the tag detection table 360 is obtained.

In S214, the position estimation unit 231 estimates the position of the sterilization target associated with the tag ID indicated by the reading result record obtained in S211 on the basis of the known position of the position tag 40 already detected by the same tag reader 100 and the relative amount of movement of the tag reader 100. For example, the position estimation unit 231 adds a vector representing the relative amount of movement of the tag reader 100 from the detection time of the position tag 40 to the detection time of the item tag 50 to the positional coordinates of the position tag 40 according to the above-described Formula (1) to derive the estimated position of the sterilization target. Next, in S215, the position estimation unit 231 estimates the area 10 where the sterilization target exists on the basis of the derived estimated position. Next, in S216, the position estimation unit 231 updates the position information stored in the target table 310 of the sterilization DB 220 on the basis of the estimation result in S214 and S215. Then, the process returns to S211.

In S217, the sterilization management unit 232 determines whether there exists a sterilization target detected by the same tag reader 100 at a time close (with a time difference less than the threshold) to the reading time indicated by the reading result record obtained in S211 by referencing the tag detection table 360. In a case where there exists no such sterilization target, the process returns to S211. In a case where such a sterilization target does exist, in other words, in a case where a sterilization target detected at roughly the same time as the detection of the sterilization tag 80 by the same tag reader 100 exists, in order to associate the sterilization target with a sterilization process, the process proceeds to S218. In S218, the sterilization management unit 232 identifies the sterilization process to be associated with the sterilization target by comparing the reading time of the two tag IDs with the processing period of each sterilization process indicated by the sterilization-related information from the sterilizer 60 in which the detected sterilization tag 80 is installed. Next, in S219, the sterilization management unit 232 associates the sterilization process identified in S218 with the corresponding sterilization target in the target table 310. Next, in S220, the sterilization management unit 232 updates the sterilization status and the sterilization deadline of the corresponding sterilization target in the target table 310. Then, the process returns to S211.

### <5-3. Notification Process (Management Server)>

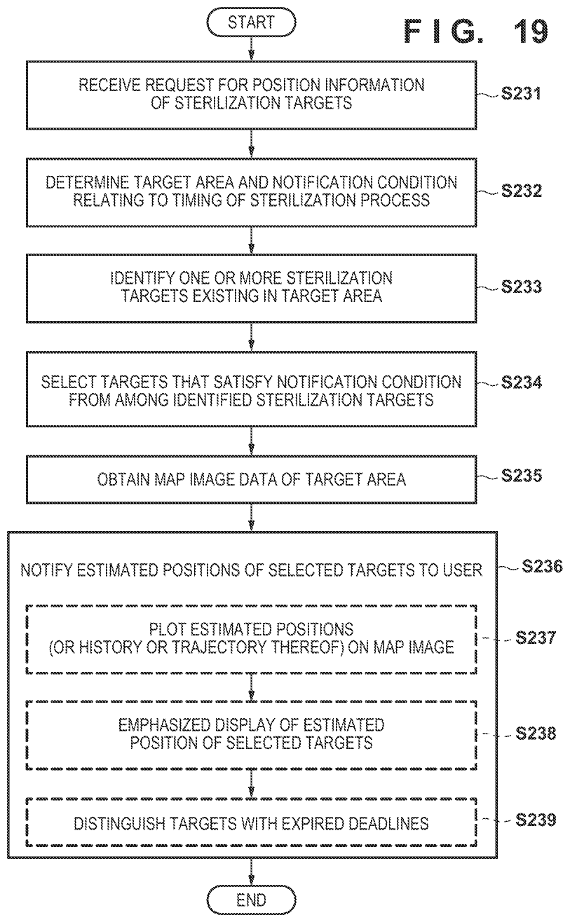

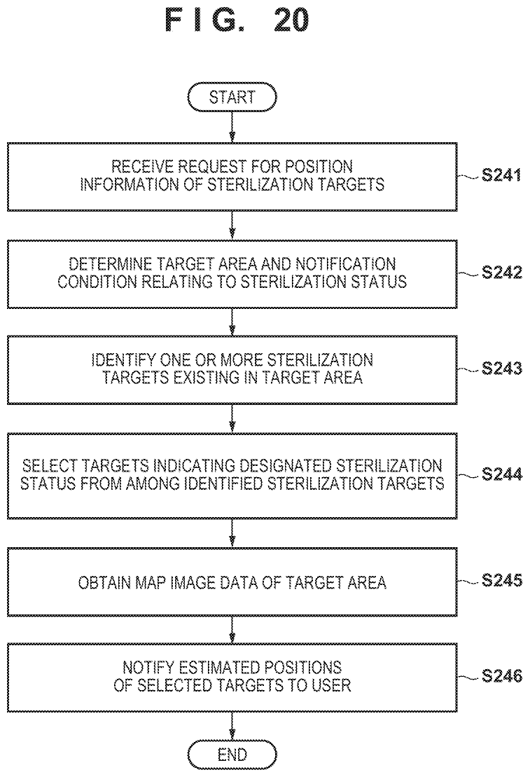

FIGS. 19 and 20 are flowcharts illustrating examples of the flow of the notification process performed by the management server 200. These notification processes may be performed when an application for sterilization management that can run on the terminal apparatus 90 accesses the management server 200, for example.

### (1) First Example

In the first example illustrated in FIG. 19, a condition relating to the timing of the sterilization process is designated as a notification condition for position information notification.

First, in S231, the notification unit 233 receives a request for position information of sterilization targets from the terminal apparatus 90. The request for position information may be received together with a notification condition designated by the user 20 via the GUI described using FIGS. 11 to 15, for example.

Next, in S232, the notification unit 233 determines the notification target area and the designated notification condition. The target area may be an area explicitly designated by the user 20 or may be an area where the terminal apparatus 90 from which the request has been transmitted is currently located. The notification condition may be designated in the format of the number of remaining days until the deadline of sterilization process.

Next, in S233, the notification unit 233 identifies one or more sterilization targets estimated to exist in the notification target area by referencing the target table 310. Next, in S234, the notification unit 233 selects, from among the identified sterilization targets, sterilization targets of which deadlines of sterilization processes satisfy the designated notification condition (for example, the sterilization targets for which number of remaining days until their deadlines of sterilization processes are equal to or less than the threshold). Also, in S235, the notification unit 233 obtains the map image data of the notification target area from the area table 320.

Then, in step S236, the notification unit 233 notifies the user 20 of the estimated positions of the sterilization targets selected in S234 via the terminal apparatus 90. The notification here may be performed in various modes. For example, in S237, the notification unit 233 displays the map image of the target area on the display of the terminal apparatus 90 and plots the estimated positions (or the history or trajectory of the estimated positions) of the selected sterilization targets on the map image. Additionally or alternatively, in S238, the notification unit 233 displays the estimated positions of the selected sterilization targets in an emphasized manner compared to estimated positions of other sterilization targets. Additionally or alternatively, in S239, the notification unit 233 displays the estimated positions of sterilization targets with expired effective periods in a manner that those sterilization targets are distinguished from the other sterilization targets.

### (2) Second Example

In the second example illustrated in FIG. 20, a condition relating to the sterilization status is designated as a notification condition for position information notification.

First, in S241, the notification unit 233 receives a request for position information of sterilization targets from the terminal apparatus 90. The request for position information may be received together with a notification condition designated by the user 20 via the GUI described using FIGS. 11 to 15, for example.

Next, in S242, the notification unit 233 determines the notification target area and the designated notification condition. The notification condition may be designated in a format for designating one or more from among candidates for the sterilization status, for example.

Next, in S243, the notification unit 233 identifies one or more sterilization targets estimated to exist in the notification target area by referencing the target table 310. Next, in S244, the notification unit 233 selects, from among the identified sterilization targets, sterilization targets that satisfies the designated notification condition (for example, the sterilization targets of which sterilization statuses indicate the designated value). Also, in S245, the notification unit 233 obtains the map image data of the notification target area from the area table 320.

Then, in step S246, the notification unit 233 notifies the user 20 of the estimated positions of the sterilization targets selected in S244 via the terminal apparatus 90. The notification here may be performed in various modes such as those described above in relation to S236 of FIG. 19.

The notification process described using FIGS. 19 and 20 is not limited to the detailed process order indicated by the flowchart and may be interactively performed between the user 20 and the management server 200 via the UI provided by the terminal apparatus 90.

### 6. Summary

Various embodiments and alteration examples of the technology according to the present disclosure have been described above in detail using FIGS. 1 to 20. According to the embodiments described above, in the sterilization management system, a first wireless device (position tag) storing first identification information is installed at a known position, and a second wireless device (item tag) storing second identification information is attached to a target for a sterilization process. A reading apparatus (tag reader) that moves together with a mobile object attempts to read the identification information from the wireless devices and measures the relative amount of movement from a reference position using a self-localization technique. The position of the target for a sterilization process is estimated on the basis of the relative amount of movement of the reading apparatus from a first point in time at which the first identification information has been read from the first wireless device by the reading apparatus to a second point in time at which the second identification information has been read from the second wireless device by the reading apparatus. The database of the sterilization management system stores position information indicating the estimated position of each target estimated in this manner and the sterilization-related information for each target. Then, at least one target for sterilization process is selected on the basis of the sterilization-related information, and the user is notified of the position where the selected target is estimated to exist on the basis of the position information stored in the database. According to this configuration, in a situation where there is a possibility of the sterilization target moving to any place within a space for users' activities, the system can continuously track the position of the sterilization target and guide the user to the estimated position. The positional range within which the sterilization target can be detected is not limited to a fixed range and may be expanded to a range within which the reading apparatus is likely to move together with the mobile object. Thus, even if the sterilization target moves to a normally unexpected place, the user can efficiently find the sterilization target on the basis of the estimated position of the sterilization target notified by the system. As a result, sterilization management can be optimized, the hygiene environment is improved, and the risk of infection can be reduced.

Also, according to the embodiments described above, the sterilization-related information may include timing information indicating the timing of when to perform the sterilization process for the sterilization target, and the user may be notified of the estimated position of the sterilization target selected on the basis of the timing information. Thus, the user can perform the sterilization process in a timely manner without deviating from the timing at which each sterilization target should be sterilized. The timing information included in the sterilization-related information may include at least one of: a sterilization process date representing a date on which the sterilization process was last performed on the sterilization target; and a deadline representing the date on which an effective period from the sterilization process date expires. Thus, it is possible to cause the user to use only sterilization targets with little or no risk from hygiene perspective in terms of their sterilization effective periods, or to prompt the user to perform the sterilization process by notifying locations of the sterilization targets with a high risk. For example, for a sterilization target that satisfies the notification condition of the number of remaining days until the sterilization process deadline being equal to or less than a preset threshold or a threshold set by the user, the user may be notified of its estimated position. Accordingly, it is possible to prevent a situation in which the effective period of sterilization expires without the necessary sterilization process being performed.

Also, according to the embodiments described above, the sterilization-related information may include status information indicating the sterilization state of the sterilization target, and the user may be notified of the estimated position of the sterilization target selected on the basis of the status information. Accordingly, the user can quickly find and use a sterilization target in a good sterilization state or can appropriately determine a sterilization target not to use due to the sterilization state being not good.

Also, according to the embodiments described above, the sterilization-related information may be output by the sterilizer that performs the sterilization process, and the sterilization-related information may be stored in a database. By the sterilization management system and the sterilizer coordinating in this manner, information such as the sterilization status and sterilization deadline of each sterilization target can be automatically collected and easily utilized for assisting in sterilization management. According to a practical example, a third wireless device (sterilization tag) storing third identification information is installed in the sterilizer. Then, the association between the sterilization-related information output from the sterilizer and the sterilization target may be determined on the basis of a correlation between a third point in time at which the third identification information has been read from the third wireless device by the reading apparatus and a fourth point in time at which the second identification information has been read from the second wireless device. In this case, for automatically associating the sterilization-related information with the sterilization target, it is sufficient that the sterilizer has a function for externally outputting the sterilization-related information. Thus, a mechanism for assisting in the sterilization management described above can be realized while keeping the cost of modifying existing sterilizers to a minimum.

Also, according to the embodiments described above, each wireless device is an RFID tag, and the reading apparatus reads the information sent back from the RFID tag utilizing energy of electromagnetic waves emitted in a reading range. In this case, a battery or a complex transceiver is not required to be installed in the wireless device attached to each target, and even in a situation where many sterilization targets are in the system, the mechanism according to the embodiments described above can be incorporated at a low cost. Also, by using a combination of RFID tags and a self-localization technique, even in environments such as indoors or underground in a closed-off environment where remote communications with an external environment is unavailable or an environment where use of strong electromagnetic waves is prohibited, the data for target position estimation can be collected. The transmission to the server of the collected data in this case may be collectively performed after the reading apparatus is moved to a place where remote communications is permitted.

Note that in the present specification, an example in which the technology according to the present disclosure is used to assist in sterilization management has been mainly described. However, the technology according to the present disclosure may also be applied to any applications other than sterilization management. Generally speaking, the technology according to the present disclosure can be applied to various types of deadline management applications in which an item with a set deadline for a certain user action that requires position tracking because of a possibility of moving within a space is treated as a target. In other words, the technology according to the present disclosure may be broadly used to enhance efficiency of deadline management for a target with a possibility of moving to any place within a space for users' activity.

### 7. Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

According to the present invention, it will be possible to realize more efficient sterilization management of sterilization targets that may move to any place within a space for users' activities.

1. A sterilization management system, comprising:
a first wireless device installed at a known position and storing first identification information;
a second wireless device attached to a target of a sterilization process and storing second identification information;
a reading apparatus that moves together with a mobile object, the reading apparatus being capable of reading, from a wireless device, identification information stored in the wireless device, and capable of measuring a relative amount of movement from a reference position using a self-localization technique;
a position estimation unit configured to estimate a position of the target based on the relative amount of movement of the reading apparatus from a first point in time at which the first identification information has been read from the first wireless device by the reading apparatus to a second point in time at which the second identification information has been read from the second wireless device by the reading apparatus;
a database that stores position information representing an estimated position of the target and sterilization-related information for the target; and
a notification unit configured to, when the target is selected based on the sterilization-related information, notify a user of a position at which the target is estimated to exist based on the position information stored in the database.

2. The sterilization management system according to claim 1, wherein the sterilization-related information includes timing information representing a timing of the sterilization process to be performed for the target, and
the notification unit is configured to select the target based on the timing information included in the sterilization-related information.

3. The sterilization management system according to claim 1, wherein the sterilization-related information includes status information representing a sterilization state for the target, and
the notification unit is configured to select the target based on the status information included in the sterilization-related information.

4. The sterilization management system according to claim 2, wherein the timing information includes at least one of a sterilization process date representing a date on which the sterilization process was previously performed for the target, and a deadline representing a date on which an effective period from the sterilization process date ends, and
the notification unit is configured to, when the deadline determined based on the timing information for the target satisfies a notification condition, select the target to notify the user of a position at which the target is estimated to exist.

5. The sterilization management system according to claim 4, wherein the notification condition includes that a number of remaining days until the deadline is equal to or less than a threshold that is preset or designated by the user.

6. The sterilization management system according to claim 4, wherein the notification unit is configured to
notify the user of a position at which the target is estimated to exist in a first notification mode for the target with the deadline not satisfying the notification condition, and
notify the user of a position at which the target is estimated to exist in a second notification mode for the target with the deadline satisfying the notification condition, the second notification mode being more emphasized than the first notification mode.

7. The sterilization management system according to claim 4, wherein the notification unit is configured to notify the user of a position at which the target is estimated to exist for the target with the deadline being a past date in a different notification mode than that for another target.

8. The sterilization management system according to claim 1, wherein the notification unit is configured to perform the notification by causing a terminal apparatus of the user to display, on a screen, a map image of an area in which the target exists with the position of the target being plotted thereon.

9. The sterilization management system according to claim 8, wherein the position information represents a plurality of estimated positions of the target that correspond respectively to a plurality of points in time, and
the notification unit is configured to cause a history of the position at which the target is estimated to exist or a trajectory of movement of the target to be plotted on the map image and displayed on the screen based on the position information.

10. The sterilization management system according to claim 8, wherein the position estimation unit is configured to further estimate a position of the reading apparatus based on the relative amount of movement measured by the reading apparatus, and
the notification unit is configured to cause the estimated position of the reading apparatus to be further plotted on the map image and displayed on the screen.

11. The sterilization management system according to claim 1, wherein the position information represents a plurality of estimated positions of the target that correspond respectively to a plurality of points in time, the plurality of estimated positions being estimated by the position estimation unit based on results of reading identification information from a wireless devices by a plurality of reading apparatuses, and on results of measuring relative amounts of movement by the plurality of reading apparatuses.

12. The sterilization management system according to claim 1, further comprising:
a management unit configured to obtain the sterilization-related information output by a sterilizer that performs the sterilization process and cause the database to store it.

13. The sterilization management system according to claim 12, further comprising:
a third wireless device installed on the sterilizer and storing third identification information;
wherein the management unit is configured to associate the sterilization-related information output by the sterilizer with the target based on correlation between a third point in time at which the third identification information has been read from the third wireless device by the reading apparatus and a fourth point in time at which the second identification information has been read from the second wireless device by the reading apparatus.

14. The sterilization management system according to claim 1, wherein the wireless devices are radio frequency identification (RFID) tags, and
the reading apparatus is configured to emit an electromagnetic wave to a reading range and read information sent back from the wireless device utilizing energy of the electromagnetic wave.

15. A sterilization management method comprising:
reading, by a reading apparatus that moves together with a mobile object, first identification information from a first wireless device installed at a known position;
reading, by the reading apparatus, second identification information from a second wireless device attached to a target of a sterilization process;
measuring, by the reading apparatus, a relative amount of movement from a reference position using a self-localization method;
estimating a position of the target based on the relative amount of movement of the reading apparatus from a first point in time at which the first identification information has been read from the first wireless device by the reading apparatus to a second point in time at which the second identification information has been read from the second wireless device by the reading apparatus;
causing a database to store position information representing an estimated position of the target and sterilization-related information for the target; and
when the target is selected based on the sterilization-related information, notifying a user of a position at which the target is estimated to exist based on the position information stored in the database.

16. An information processing apparatus comprising:
a communication unit configured to receive, from a reading apparatus that is capable of reading identification information stored in a wireless device from the wireless device and capable of measuring a relative amount of movement from a reference position using a self-localization technique, a result of reading the identification information and a result of measuring the relative amount of movement;
a position estimation unit configured to estimate a position of a target of a sterilization process based on the relative amount of movement of the reading apparatus from a first point in time at which first identification information has been read by the reading apparatus from a first wireless device installed at a known position to a second point in time at which second identification information has been read by the reading apparatus from a second wireless device attached to the target;
a database that stores position information representing an estimated position of the target and sterilization-related information for the target; and
a notification unit configured to, when the target is selected based on the sterilization-related information, notify a user of a position at which the target is estimated to exist based on the position information stored in the database.
